(19) United States
(12) Patent Application Publication
(10) Pub. No.: US 2021/0259586 A1
(43) Pub. Date: Aug. 26, 2021

(54) MEASUREMENT APPARATUS AND BIOLOGICAL INFORMATION MEASUREMENT APPARATUS

(71) Applicant: Ricoh Company, Ltd., Tokyo (JP)

(72) Inventors: Yoshihiro OBA, Miyagi (JP); Ryosuke KASAHARA, Kanagawa (JP); Yoshio WADA, Miyagi (JP); Toshihide SASAKI, Miyagi (JP)

(73) Assignee: Ricoh Company, Ltd., Tokyo (JP)

(21) Appl. No.: 17/162,010

(22) Filed: Jan. 29, 2021

(30) Foreign Application Priority Data
Feb. 20, 2020 (JP) 2020-027444

(51) Int. Cl. A61B 5/1491; A61B 5/145; A61B 5/1455

(52) U.S. Cl. CPC A61B 5/1491; A61B 2562/0271; A61B 5/1455; A61B 5/14532

(57) ABSTRACT

A measurement apparatus includes a total reflection member configured to totally reflect an incoming probe beam in a state being in contact with a measured object; and a temperature adjuster configured to maintain, to a predetermined temperature, a temperature of a contact region of the total reflection member with the measured object.

### CROSS-REFERENCE TO RELATED APPLICATIONS

This patent application is based on and claims priority pursuant to 35 U.S.C. § 119(a) to Japanese Patent Application No. 2020-027444, filed on Feb. 20, 2020, in the Japan Patent Office, the entire disclosure of which is hereby incorporated by reference herein.

### BACKGROUND

### Technical Field

The present disclosure relates to a measurement apparatus and a biological information measurement apparatus.

### Related Art

In recent years, the number of diabetics has increased worldwide, and non-invasive blood glucose measurement without blood sampling is desired.

Various methods for measuring biometric information such as a blood glucose level using light have been proposed, including methods using near-infrared light, methods using mid-infrared light, and methods using Raman spectroscopy. In the mid-infrared range, which is a fingerprint range with a large absorption of glucose, the sensitivity of measurement can be made higher than that in the near-infrared range.

A light-emitting device such as a quantum cascade laser (QCL) is available as a light source in the mid-infrared range. Such a light-emitting device requires a number of laser light sources corresponding to the number of wavelengths to be used. From the viewpoint of a reduction in apparatus size, it is desirable to limit the number of wavelengths in mid-infrared range to several wavelengths.

For accurate measurement of glucose concentration in a specific wavelength region such as the mid-infrared range by using the attenuated total reflection (ATR) method, a method using wavelengths corresponding to absorption peaks of glucose (1035 cm⁻¹, 1080 cm⁻¹, and 1110 cm⁻¹) has been proposed.

### SUMMARY

According to an embodiment of the present disclosure, a measurement apparatus includes a total reflection member configured to totally reflect an incoming probe beam in a state being in contact with a measured object; and a temperature adjuster configured to maintain, to a predetermined temperature, a temperature of a contact region of the total reflection member with the measured object.

According to another embodiment of the present disclosure, a biological information measurement apparatus includes a total reflection member configured to totally reflect an incoming probe beam in a state being in contact with a live subject to be measured; and a temperature adjuster configured to maintain, to a predetermined temperature, a temperature of a contact region of the total reflection member with the live subject.

### BRIEF DESCRIPTION OF THE SEVERAL VIEWS OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an example general arrangement of a blood glucose measurement apparatus according to embodiments;

FIG. 2 illustrates the operation of an attenuated total reflection (ATR) prism of a measurement device of the blood glucose measurement apparatus illustrated in FIG. 1;

FIG. 3 is a perspective view illustrating the structure of the ATR prism illustrated in FIG. 2;

FIG. 4 is a perspective view illustrating the structure of a hollow optical fiber of the measurement device illustrated in FIG. 1;

FIG. 5 is a block diagram of an example hardware configuration of a processor of the blood glucose measurement apparatus illustrated in FIG. 1;

FIG. 6 is a block diagram illustrating an example functional configuration of the processor illustrated in FIG. 5;

FIGS. 7A, 7B, and 7C illustrate an example operation of switching a probe beam to be used among first, second, and third probe beams by using a shutter control unit illustrated in FIG. 6;

FIG. 8 is a flowchart illustrating an example operation of the blood glucose measurement apparatus illustrated in FIG. 1;

FIG. 9A illustrates the light intensities of the probe beams in a comparative example;

FIG. 9B illustrates the light intensities of the probe beams that are changed in three or more stages by using an absorbance acquisition unit of the processor illustrated in FIG. 6;

FIGS. 10A, 10B, 10C, and 10D illustrate an example of correction of a displacement of a probe beam;

FIG. 11A illustrates total reflection of the probe beams when an incidence surface of the ATR prism illustrated in FIG. 2 is a flat surface;

FIG. 11B illustrates total reflection of the probe beams when the incidence surface is a diffusion surface;

FIG. 11C illustrates an incidence surface of the diffusion surface;

FIG. 11D illustrates an incidence surface of a concave surface;

FIG. 11E illustrates an incidence surface of a convex surface;

FIGS. 12A, 12B, and 12C illustrate changes in relative position between the ATR prism and first and second hollow optical fibers;

FIG. 13 illustrates supporting members of the first and second hollow optical fibers and the ATR prism of the measurement device illustrated in FIG. 1;

FIG. 14A illustrates a light source drive current in a comparative example;

FIG. 14B illustrates a high-frequency modulated light source drive current;

FIGS. 15A and 15B are a front view and a side view of a blood glucose measurement apparatus according to a first embodiment, respectively;

FIG. 16 illustrates a contact region between the ATR prism and the lips of a subject;

FIGS. 17A and 17B are a front view and a side view of a blood glucose measurement apparatus according to a second embodiment, respectively;

FIG. 17C is a perspective view of the blood glucose measurement apparatus according to the second embodiment as viewed from one side;

FIG. 17D is a perspective view of the blood glucose measurement apparatus according to the second embodiment as viewed from another side;

FIG. 18 is a block diagram of an example functional configuration of a processor according to the second embodiment;

FIG. 19 illustrates changes in temperature sensor output over time according to a comparative example;

FIG. 20 illustrates changes in temperature sensor output over time according to the second embodiment;

FIGS. 21A, 21B, and 21C are a front view, a side view, and a perspective view of a blood glucose measurement apparatus according to a third embodiment, respectively;

FIGS. 22A and 22B are a front view and a side view of a blood glucose measurement apparatus according to a first modification, respectively;

FIGS. 23A and 23B are a front view and a side view of a blood glucose measurement apparatus according to a second modification, respectively;

FIGS. 24A and 24B are a front view and a side view of a blood glucose measurement apparatus according to a third modification, respectively;

FIGS. 25A and 25B are a front view and a side view of a blood glucose measurement apparatus according to a fourth modification, respectively;

FIGS. 26A and 26B are a front view and a side view of a blood glucose measurement apparatus according to a fifth modification, respectively; and

FIGS. 27A and 27B are a front view and a side view of a blood glucose measurement apparatus according to a sixth modification, respectively.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this patent specification is not intended to be limited to the specific terminology so selected, and it is to be understood that each specific element includes all technical equivalents that have the same function, operate in a similar manner, and achieve a similar result.

Referring now to the drawings, wherein like reference numerals designate identical or corresponding parts throughout the several views thereof, embodiments of this disclosure are described. As used herein, the singular forms “a,” “an,” and “the” are intended to include the plural forms as well, unless the context clearly indicates otherwise.

### Terms in Embodiments

The term “mid-infrared range” refers to a wavelength region of 2 to 14 μm and is an example of a specific wavelength region.

The term “probe beam” refers to light to be used for absorbance measurement and biometric information measurement. In embodiments, the term “probe beam” corresponds to light totally reflected by a total reflection member, attenuated by a live subject, and then detected by a light intensity detector.

The term “attenuated total reflection (ATR) method” refers to a method for acquiring an absorption spectrum of an object to be measured by using a field (evanescent wave) penetrated from a total reflection surface when total reflection occurs in a total reflection member such as an ATR prism disposed in contact with the object to be measured.

The term “absorbance” refers to a dimensionless quantity indicating how much the light intensity is decreased when light passes through an object. In embodiments, attenuation of the field penetrated from the total reflection surface by a live subject is measured as absorbance by using the ATR method.

The term “blood glucose level” refers to the concentration of dextrose (glucose) contained in blood.

In embodiments, the term “detection value” refers to a detection value obtained by a light intensity detector.

For the term “wave number,” the relationship between a wavelength λ (μm) and a wave number k (cm⁻¹) satisfies k=10000/λ.

Hereinafter, embodiments will be described by taking, as an example, a blood glucose measurement apparatus (an example of a biometric information measurement apparatus) that measures a blood glucose level (an example of biometric information) based on an absorbance measured using an ATR prism (an example of a total reflection member).

### EMBODIMENTS

First, a blood glucose measurement apparatus 100 according to embodiments will be described.

In embodiments, a plurality of probe beams having different wavelengths in the mid-infrared range are made incident on a total reflection member in contact with a live subject, the absorbance of the plurality of probe beams is acquired based on the ATR method, and a blood glucose level is measured based on the acquired absorbance.

Example General Arrangement of Blood Glucose Measurement Apparatus

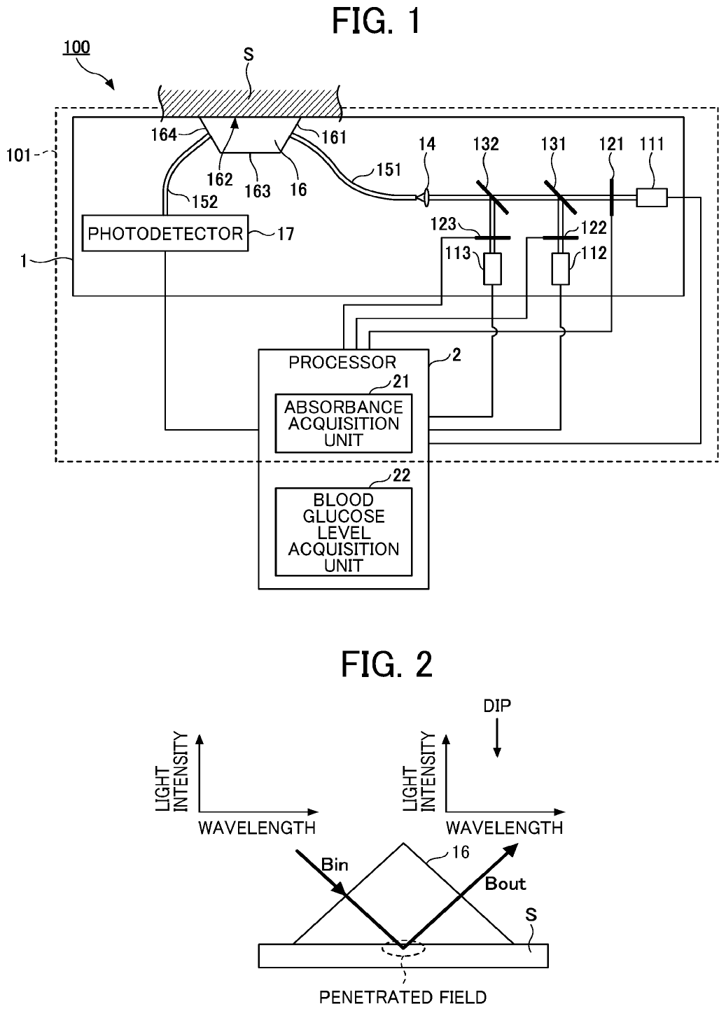

FIG. 1 illustrates an example general arrangement of the blood glucose measurement apparatus 100 according to embodiments. As illustrated in FIG. 1, the blood glucose measurement apparatus 100 includes a measurement device 1 and a processor 2.

The measurement device 1 is an optical head configured to perform the ATR method and outputs a detection signal of a probe beam attenuated by a live subject to the processor 2. The processor 2 is an information processing apparatus, such as a personal computer (PC), that acquires absorbance data based on the detection signal and acquires and outputs a blood glucose level based on the absorbance data.

The measurement device 1 includes a first light source 111, a second light source 112, a third light source 113, a first shutter 121, a second shutter 122, and a third shutter 123. The measurement device 1 further includes a first half mirror 131, a second half mirror 132, a coupling lens 14, a first hollow optical fiber 151, an ATR prism 16, a second hollow optical fiber 152, and a photodetector 17.

The processor 2 includes an absorbance acquisition unit 21 and a blood glucose level acquisition unit 22. As indicated by a broken-line box, the measurement device 1 and the absorbance acquisition unit 21 together construct an absorbance measurement apparatus 101.

In the measurement device 1, the first light source 111, the second light source 112, and the third light source 113 are each a quantum cascade laser electrically connected to the processor 2 and configured to emit a laser beam in the mid-infrared range in accordance with a control signal from the processor 2.

In embodiments, the first light source 111 emits a laser beam having a wave number of 1050 cm⁻¹ as a first probe beam, the second light source 112 emits a laser beam having a wave number of 1070 cm⁻¹ as a second probe beam, and the third light source 113 emits a laser beam having a wave number of 1100 cm⁻¹ as a third probe beam.

The laser beams having wave numbers of 1050 cm⁻¹, 1070 cm⁻¹, and 1100 cm⁻¹ correspond to the wave numbers of absorption peaks of glucose. These wave numbers are used to measure the absorbance to provide accurate measurement of glucose concentration based on the absorbance.

The first shutter 121, the second shutter 122, and the third shutter 123 are each an electromagnetic shutter electrically connected to the processor 2 and controlled to be opened or closed in accordance with a control signal from the processor 2.

When the first shutter 121 is opened, the first probe beam from the first light source 111 passes through the first shutter 121 and reaches the first half mirror 131. On the other hand, when the first shutter 121 is closed, the first probe beam is blocked by the first shutter 121 and does not reach the first half mirror 131.

When the second shutter 122 is opened, the second probe beam from the second light source 112 passes through the second shutter 122 and reaches the first half mirror 131. On the other hand, when the second shutter 122 is closed, the second probe beam is blocked by the second shutter 122 and does not reach the first half mirror 131.

When the third shutter 123 is opened, the third probe beam from the third light source 113 passes through the third shutter 123 and reaches the second half mirror 132. On the other hand, when the third shutter 123 is closed, the third probe beam is blocked by the third shutter 123 and does not reach the second half mirror 132.

The first half mirror 131 and the second half mirror 132 are each an optical element for transmitting a portion of incident light and reflecting the rest. Such an optical element can be a transmissive substrate provided with an optical thin film that allows a portion of incident light to pass through and reflects the rest of the incident light.

However, the optical element is not limited to an optical thin film and may be a transmissive substrate provided with a diffraction structure that allows a portion of incident light to pass through and reflects (or diffracts) the rest. The diffraction structure suppresses light absorption, which is preferable.

The first half mirror 131 transmits the first probe beam passing through the first shutter 121 and reflects the second probe beam passing through the second shutter 122. The second half mirror 132 transmits the first probe beam and the second probe beam and reflects the third probe beam passing through the third shutter 123.

In each of the first half mirror 131 and the second half mirror 132, preferably, the light intensity ratio of transmitted light to reflected light is set to substantially 1:1. However, the light intensity ratios may be adjusted in accordance with the light intensities of the probe beams emitted from the respective light sources or the like.

The first to third probe beams that have passed through the first half mirror 131 or the second half mirror 132 are guided into the first hollow optical fiber 151 via the coupling lens 14, propagated through the first hollow optical fiber 151, and guided into the ATR prism 16 via an incidence surface 161 of the ATR prism 16.

The ATR prism 16 is an optical prism that propagates the first to third probe beams incident from the incidence surface 161 toward an emission surface 164 while totally reflecting the first to third probe beams and emits the first to third probe beams from the emission surface 164. The ATR prism 16 is an example of a total reflection member. As illustrated in FIG. 1, the ATR prism 16 is disposed such that a first total reflection surface 162 is in contact with a live subject S (sample), which is an example of an object to be measured (measured object).

The first to third probe beams guided into the ATR prism 16 are repeatedly totally reflected on the first total reflection surface 162 and a second total reflection surface 163 facing the first total reflection surface 162, and are guided into the second hollow optical fiber 152 via the emission surface 164.

The first to third probe beams guided through the second hollow optical fiber 152 reach the photodetector 17. The photodetector 17 is a detector capable of detecting light having a wavelength in the mid-infrared range and is configured to photoelectrically convert the received first to third probe beams and output electrical signals corresponding to the light intensities to the processor 2 as detection signals. Examples of the photodetector 17 include an infrared photodiode (PD), a mercury cadmium telluride (MCT) detection element, and a bolometer. The photodetector 17 is an example of a light intensity detector. In the following, the first to third probe beams are sometimes referred to simply as probe beams when not distinguished from one another.

The processor 2 is an information processing apparatus such as a PC. The absorbance acquisition unit 21 of the processor 2 acquires absorbance data of the respective probe beams based on the detection signals of the photodetector 17 and outputs the absorbance data to the blood glucose level acquisition unit 22. The blood glucose level acquisition unit 22 acquires blood glucose level data of the live subject S based on the absorbance data of the probe beams.

In FIG. 1, the measurement device 1 is surrounded by a solid-line frame, and the absorbance measurement apparatus 101 is surrounded by a broken-line frame to indicate the configuration of the measurement device 1 and the components of the absorbance measurement apparatus 101. Note that these frames do not depict housings. The ATR prism 16 is not accommodated in a housing, and at least one of the first total reflection surface 162 or the second total reflection surface 163 can be brought into contact with any portion of the live subject S.

ATR Prism

Next, referring to FIG. 2, the operation of the ATR prism 16 will be described. As illustrated in FIG. 2, the ATR prism 16 of the measurement device 1 is disposed in contact with the live subject S. The probe beams incident on the ATR prism 16 are attenuated according to the infrared absorption spectrum of the live subject S. The attenuated probe beams are received by the photodetector 17, and the respective light intensities of the probe beams are detected. The detection signals are input to the processor 2, and the processor 2 acquires and outputs absorbance data and blood glucose level data based on the detection signals.

An infrared ATR method is effective for detection by spectroscopy in the mid-infrared range where the intensity of light absorbed by glucose is obtained. The infrared ATR method utilizes “penetration” of a field occurring when the probe beams, which are infrared light, are made incident on the ATR prism 16 having a high refractive index and total reflection occurs at the interface between the ATR prism 16 and the outside (for example, the live subject S). When measurement is performed with the live subject S (an object to be measured) being in contact with the ATR prism 16, the penetrated field is absorbed by the live subject S.

When infrared light having a wide wavelength range of 2 to 12 m is used as the probe beams, light having a wavelength caused by molecular vibration energy of the live subject S is absorbed. Then, light absorption appears as a dip at the corresponding wavelength of the probe beams transmitted through the ATR prism 16. This method enable acquisition of a large amount of energy of the detection light transmitted through the ATR prism 16 and is advantageous, in particular, in infrared spectroscopy using probe beams with weak power.

When infrared light is used, the light penetrates into the live subject S from the ATR prism 16 at a depth of about several micrometers and does not reach capillaries at a depth of about several hundreds of microns. However, it is known that components such as blood plasma in blood vessels leak into the skin or mucosal cells as tissue fluid (interstitial fluid). Detection of the glucose component present in the tissue fluid enables measurement of the blood glucose level.

The concentration of the glucose component in the tissue fluid is considered to increase as the distance to capillaries decreases, and the ATR prism 16 is constantly pressed against the object being measured at a constant pressure. In embodiments, a multiple-reflection ATR prism having a trapezoidal cross section is used as the ATR prism 16 to facilitate such pressing.

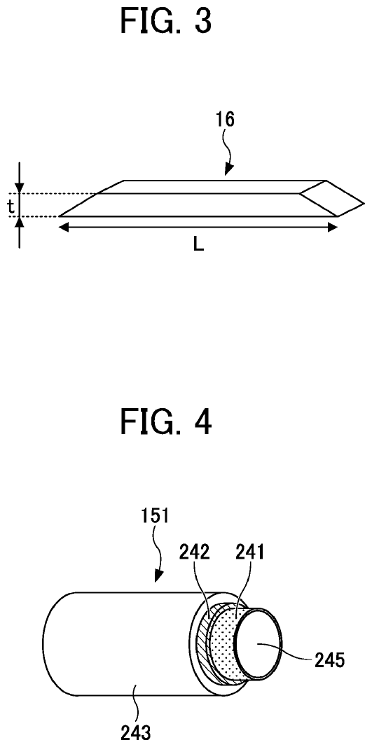

FIG. 3 is a perspective view illustrating the structure of the ATR prism 16 according to embodiments. As illustrated in FIG. 3, the ATR prism 16 is a trapezoidal prism. As the number of multiple reflections in the ATR prism 16 increases, the detection sensitivity of glucose increases. In addition, since the area of contact with the live subject S can be made large, fluctuations in detection value caused by a change in the pressure with which the ATR prism 16 is pressed can be kept small. The ATR prism 16 has a bottom surface having a length L of, for example, 24 mm. The ATR prism 16 has a thickness t that is set to be thin such as 1.6 mm or 2.4 mm so as to allow multiple reflection.

The ATR prism 16 may be made of a material that is non-toxic to the human body and exhibits high transmission characteristics at or around a wavelength of about 10 μm, which is the absorption band of glucose. In one example, among materials satisfying the conditions described above, a prism made of zinc sulfide (ZnS) with a refractive index of 2.2 and a large penetration of light to facilitate detection of deeper positions may be used for the ATR prism 16. Unlike zinc selenide (ZnSe), which is used as a typical infrared material, ZnS is indicated to be non-carcinogenic and is used as a non-toxic dye (lithopone) in dental materials.

In a typical ATR measurement apparatus, an ATR prism is secured to a relatively large apparatus. Thus, a portion of a live subject to be measured is limited to a body surface such as a fingertip or a forearm. Since the skin in such portions is covered with a stratum corneum having a thickness of about 20 μm, the glucose concentration to be detected is low. In addition, the stratum corneum is affected by secretion of sweat or sebum, resulting in limited reproducibility of measurement. Accordingly, the blood glucose measurement apparatus 100 includes the first hollow optical fiber 151 and the second hollow optical fiber 152 capable of transmitting the probe beams, which are infrared light, at low loss, and one end of each of the first hollow optical fiber 151 and the second hollow optical fiber 152 is abutted against the ATR prism 16 when in use.

One end of the first hollow optical fiber 151 is in contact with (abutting against) the ATR prism 16, thereby optically connecting the first hollow optical fiber 151 to the incidence surface 161 of the ATR prism 16. As a result, outgoing light from the first hollow optical fiber 151 is incident on the incidence surface 161 of the ATR prism 16.

One end of the second hollow optical fiber 152 is contact with (abutting against) the ATR prism 16, thereby optically connecting the second hollow optical fiber 152 to the emission surface 164 of the ATR prism 16. As a result, outgoing light from the emission surface 164 of the ATR prism 16 is guided into the second hollow optical fiber 152.

The ATR prism 16 enables measurement at an earlobe, in which capillaries are relatively close to the skin surface and which is less affected by sweat or sebum, or at the oral mucosa that lacks stratum corneum.

FIG. 4 is a perspective view illustrating an example structure of a hollow optical fiber in the blood glucose measurement apparatus 100. Mid-infrared light having a relatively long wavelength, which is used for glucose measurement, is difficult to pass through a silica glass optical fiber since the light is absorbed by glass. Various types of optical fibers for infrared transmission using special materials have been developed. However, materials that are toxic or have hygroscopicity or poor chemical durability are not desirable for use in the medical field.

In contrast, each of the first hollow optical fiber 151 and the second hollow optical fiber 152 is constructed of a tube 243 made of a harmless material such as glass or plastic, and a metal thin film 242 and a dielectric thin film 241 are disposed in this order on an inner surface of the tube 243. The metal thin film 242 is made of a low-toxicity material, such as silver, and is coated with the dielectric thin film 241 to have chemical and mechanical durability. In addition, a core 245 is air, which does not absorb mid-infrared light, and enables low-loss transmission of mid-infrared light over a wide wavelength range.

Configuration of Processor

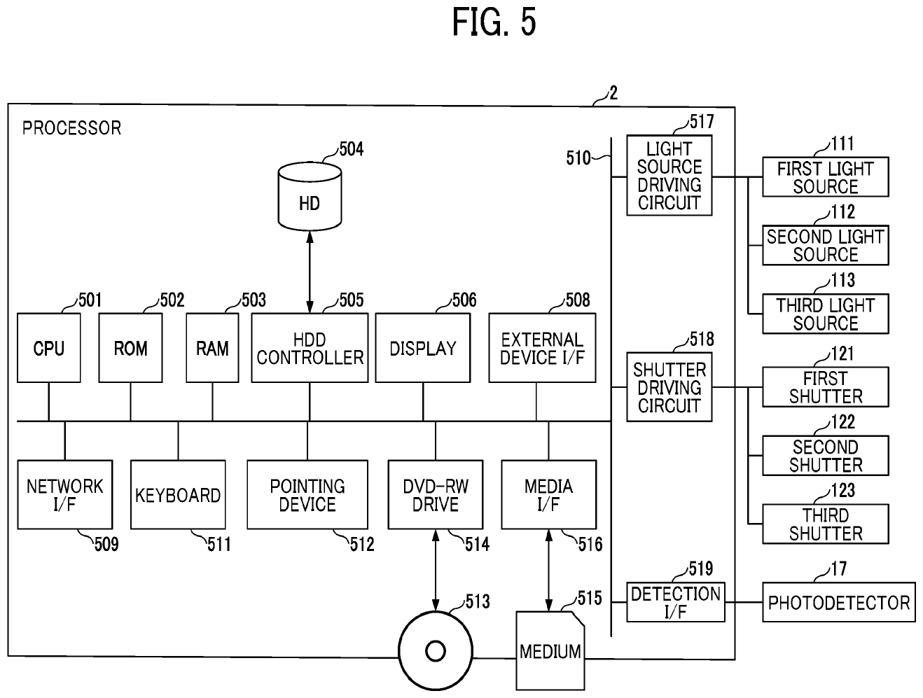

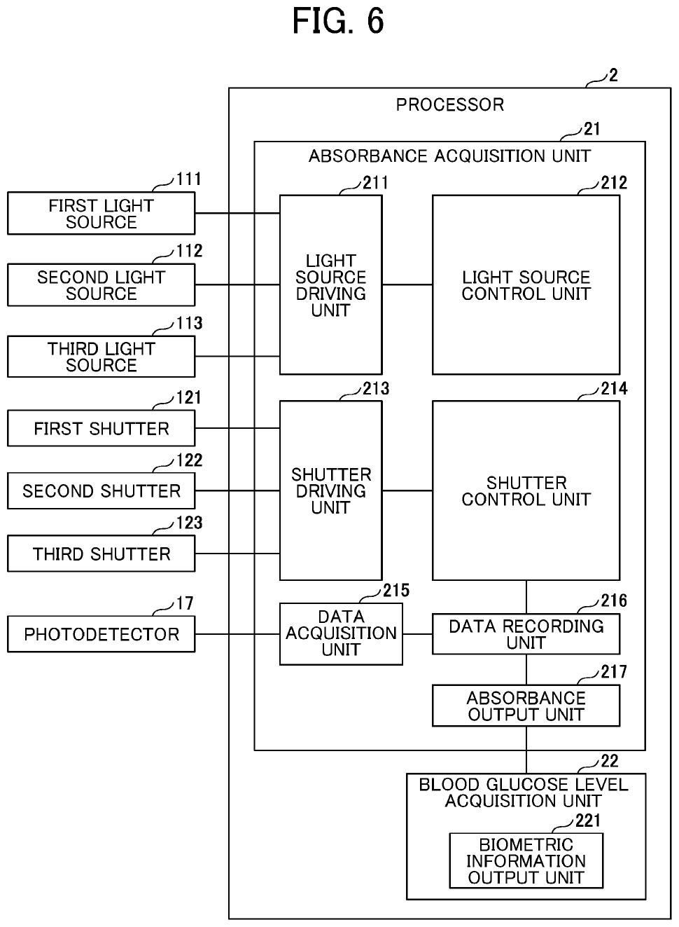

Next, referring to FIGS. 5 and 6, the configuration of the processor 2 will be described.

FIG. 5 is a block diagram illustrating an example hardware configuration of the processor 2 according to embodiments. As illustrated in FIG. 5, the processor 2 includes a central processing unit (CPU) 501, a read only memory (ROM) 502, a random access memory (RAM) 503, a hard disk (HD) 504, a hard disk drive (HDD) controller 505, and a display 506. The processor 2 further includes an external device interface (I/F) 508, a network I/F 509, a bus line 510, a keyboard 511, a pointing device 512, a digital versatile disk rewritable (DVD-RW) drive 514, a media 1/F 516, a light source driving circuit 517, a shutter driving circuit 518, and a detection I/F 519.

The CPU 501 controls the overall operation of the processor 2. The ROM 502 stores programs such as an initial program loader (IPL) to boot the CPU 501. The RAM 503 is used as a work area for the CPU 501.

The HD 504 stores various data such as a control program. The HDD controller 505 controls reading and writing of various data from and to the HD 504 under control of the CPU 501. The display 506 displays various information such as a cursor, a menu, a window, a character, or an image.

The external device I/F 508 is an interface for connecting to various external devices. Examples of the external device include, but are not limited to, a universal serial bus (USB) memory and a printer. The network I/F 509 is an interface for performing data communication using a communication network. The bus line 510 may be an address bus or a data bus, which electrically connects various elements such as the CPU 501 illustrated in FIG. 5.

The keyboard 511 is an example of an input device provided with a plurality of keys for allowing a user to input characters, numerals, or various instructions. The pointing device 512 is an example of an input device that allows a user to select or execute various instructions, select a target for processing, or move a cursor being displayed. The DVD-RW drive 514 reads and writes various data from and to a DVD-RW 513, which is an example of a removable recording medium. The removable recording medium is not limited to a DVD-RW and may be digital versatile disc-recordable (DVD-R) or the like. The media I/F 516 controls reading and writing (storing) of data from and to a recording medium 515 such as a flash memory.

The light source driving circuit 517 is an electric circuit electrically connected to the first light source 111, the second light source 112, and the third light source 113 and configured to output a drive voltage for causing the first light source 111, the second light source 112, and the third light source 113 to emit infrared light in accordance with a control signal. The shutter driving circuit 518 is an electric circuit electrically connected to the first shutter 121, the second shutter 122, and the third shutter 123 and configured to output a drive voltage for opening or closing the first shutter 121, the second shutter 122, and the third shutter 123 in accordance with a control signal.

The detection I/F 519 is an electric circuit such as an analog/digital (A/D) conversion circuit serving as an interface for acquiring a detection signal of the photodetector 17. The detection I/F 519 also has a function of an interface for acquiring detection signals of various sensors such as a pressure sensor and a temperature sensor, as well as a detection signal of the photodetector 17.

FIG. 6 is a block diagram illustrating an example functional configuration of the processor 2 according to embodiments. As illustrated in FIG. 6, the processor 2 includes the absorbance acquisition unit 21 and the blood glucose level acquisition unit 22.

The absorbance acquisition unit 21 includes a light source driving unit 211, a light source control unit 212, a shutter driving unit 213, a shutter control unit 214, a data acquisition unit 215, a data recording unit 216, and an absorbance output unit 217.

The function of the light source driving unit 211 is implemented by, for example, the light source driving circuit 517. The function of the shutter driving unit 213 is implemented by, for example, the shutter driving circuit 518. The function of the data acquisition unit 215 is implemented by, for example, the detection I/F 519. The function of the data recording unit 216 is implemented by, for example, the HD 504. The functions of the light source control unit 212, the shutter control unit 214, and the absorbance output unit 217 are implemented by, for example, the CPU 501 executing a predetermined program.

The light source driving unit 211 outputs a drive voltage based on a control signal input from the light source control unit 212 to cause each of the first light source 111, the second light source 112, and the third light source 113 to emit an infrared light beam. The light source control unit 212 controls the emission timings or light intensities of the infrared light beams by using a control signal.

The shutter driving unit 213 outputs a drive voltage based on a control signal input from the shutter control unit 214 to drive opening or closing of each of the first shutter 121, the second shutter 122, and the third shutter 123. The shutter control unit 214 controls the timing or period when each shutter is opened by using a control signal. The shutter control unit 214 is an example of an incidence controller.

The data acquisition unit 215 outputs to the data recording unit 216 detection values of light intensities acquired by sampling detection signals consecutively output from the photodetector 17 at intervals of a predetermined cycle. The data recording unit 216 records therein the detection values input from the data acquisition unit 215.

The absorbance output unit 217 executes predetermined arithmetic processing based on the detection values read from the data recording unit 216 to acquire absorbance data, and outputs the acquired absorbance data to the blood glucose level acquisition unit 22.

The absorbance output unit 217 may output the acquired absorbance data to an external device such as a PC via the external device I/F 508 or output the acquired absorbance data to an external server or the like via the network I/F 509 and a network. Alternatively, the absorbance output unit 217 may output the acquired absorbance data to the display 506 (see FIG. 5) for display.

The blood glucose level acquisition unit 22 includes a biometric information output unit 221, which is an example of an output unit. The biometric information output unit 221 executes predetermined arithmetic processing based on the absorbance data input from the absorbance acquisition unit 21 to acquire blood glucose level data, and outputs the acquired blood glucose level data to the display 506 or the like for display.

The biometric information output unit 221 may output the blood glucose level data to an external device such as a PC via the external device I/F 508 or output the blood glucose level data to an external server or the like via the network I/F 509 and a network.

Alternatively, the biometric information output unit 221 may be configured to also output the reliability of blood glucose measurement.

The processing for acquiring blood glucose level data from absorbance data may be implemented by the technique disclosed in WO/2019/039269, for example, and will not be described in detail herein.

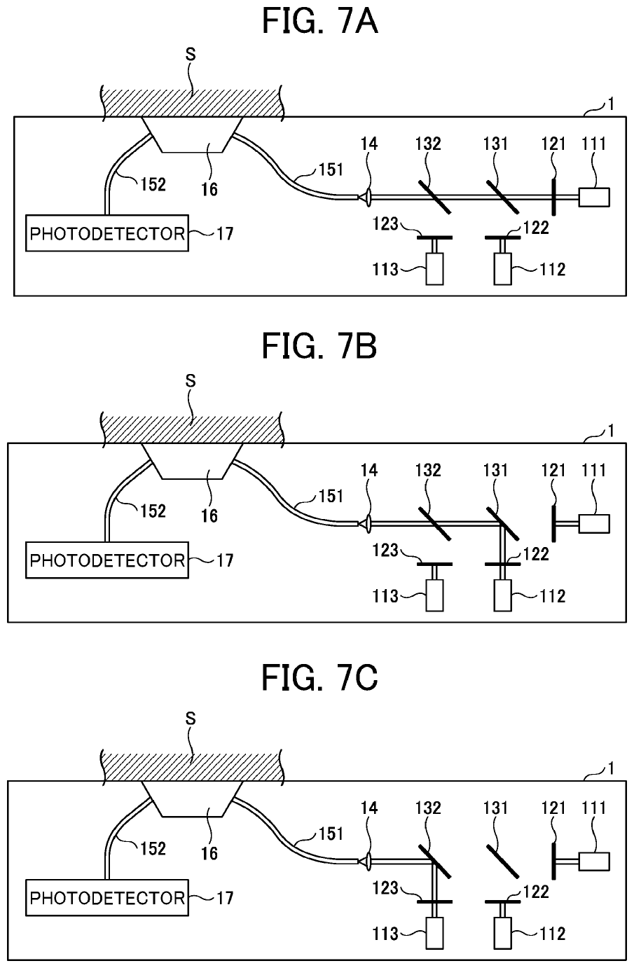

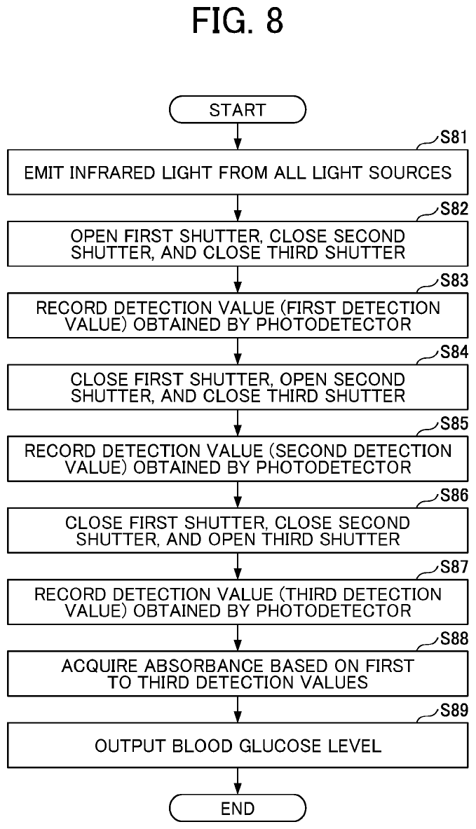

Example Operation of Blood Glucose Measurement Apparatus Next, referring to FIGS. 7 to 8, the operation of the blood glucose measurement apparatus 100 will be described.

Example Operation of Switching Probe Beams FIGS. 7A, 7B, and 7C illustrate an example operation of switching probe beams.

FIG. 7A illustrates the state of the measurement device 1 when the first probe beam is used, FIG. 7B illustrates the state of the measurement device 1 when the second probe beam is used, and FIG. 7C illustrates the state of the measurement device 1 when the third probe beam is used.

In embodiments, the probe beams emitted from the respective light sources are controlled to be incident on the ATR prism 16 by opening and closing the corresponding shutters. Accordingly, during the measurement of the absorbance and the blood glucose level, the first light source 111, the second light source 112, and the third light source 113 constantly emit infrared light beams.

In FIG. 7A, the first shutter 121 is opened in response to the control signal. The first probe beam emitted from the first light source 111 passes through the first shutter 121 and is transmitted through the first half mirror 131 and the second half mirror 132 and guided into the first hollow optical fiber 151 via the coupling lens 14. After that, the first probe beam is propagated through the first hollow optical fiber 151 and is then incident on the ATR prism 16.

Since the second shutter 122 and the third shutter 123 are closed, the second probe beam and the third probe beam are not incident on the ATR prism 16. In this state, therefore, the absorbance of the first probe beam subjected to attenuation in the ATR prism 16 is measured.

In FIG. 71B, the second shutter 122 is opened in response to the control signal. The second probe beam emitted from the second light source 112 passes through the second shutter 122 and is reflected by the first half mirror 131, transmitted through the second half mirror 132, and guided into the first hollow optical fiber 151 via the coupling lens 14. After that, the second probe beam is propagated through the first hollow optical fiber 151 and is then incident on the ATR prism 16.

Since the first shutter 121 and the third shutter 123 are closed, the first probe beam and the third probe beam are not incident on the ATR prism 16. In this state, therefore, the absorbance of the second probe beam subjected to attenuation in the ATR prism 16 is measured.

In FIG. 7C, the third shutter 123 is opened in response to the control signal. The third probe beam emitted from the third light source 113 passes through the third shutter 123 and is reflected by the second half mirror 132 and guided into the first hollow optical fiber 151 via the coupling lens 14. After that, the third probe beam is propagated through the first hollow optical fiber 151 and is then incident on the ATR prism 16.

Since the first shutter 121 and the second shutter 122 are closed, the first probe beam and the second probe beam are not incident on the ATR prism 16. In this state, therefore, the absorbance of the third probe beam subjected to attenuation in the ATR prism 16 is measured.

When all of the first shutter 121, the second shutter 122, and the third shutter 123 are closed, none of the first probe beam, the second probe beam, and the third probe beam is incident on the ATR prism 16 or reaches the photodetector 17.

As described above, the shutter control unit 214 (see FIG. 6) serving as an incidence controller controls the opening and closing of each shutter to switch between the state where the first to third probe beams are sequentially incident on the ATR prism 16 and the state where none of the first to third probe beams is incident on the ATR prism 16.

Example Operation of Blood Glucose Measurement Apparatus

FIG. 8 is a flowchart illustrating an example operation of the blood glucose measurement apparatus 100.

First, in step S81, all of the first light source 111, the second light source 112, and the third light source 113 emit infrared light beams in response to a control signal of the light source control unit 212. In this initial state, all of the first shutter 121, the second shutter 122, and the third shutter 123 are closed.

Then, in step S82, the shutter control unit 214 opens the first shutter 121 and closes the second shutter 122 and the third shutter 123.

Then, in step S83, the data recording unit 216 records therein a detection value (first detection value) obtained by the photodetector 17, which is acquired by the data acquisition unit 215.

Then, in step S84, the shutter control unit 214 opens the second shutter 122 and closes the first shutter 121 and the third shutter 123.

Then, in step S85, the data recording unit 216 records therein a detection value (second detection value) obtained by the photodetector 17, which is acquired by the data acquisition unit 215.

Then, in step S86, the shutter control unit 214 opens the third shutter 123 and closes the first shutter 121 and the second shutter 122.

Then, in step S87, the data recording unit 216 records therein a detection value (third detection value) obtained by the photodetector 17, which is acquired by the data acquisition unit 215.

Then, in step S88, the absorbance output unit 217 acquires absorbance data of the first to third probe beams based on the first to third detection values and outputs the absorbance data to the biometric information output unit 221.

Then, in step S89, the biometric information output unit 221 executes predetermined arithmetic processing based on the absorbance data of the first to third probe beams to acquire blood glucose level data, and outputs the acquired blood glucose level data to the display 506 (see FIG. 5) for display.

As described above, the blood glucose measurement apparatus 100 can acquire and output blood glucose level data.

In the above-described embodiments, as a non-limiting example, the first shutter 121, the second shutter 122, and the third shutter 123, which are electromagnetic shutters, are controlled to switch incidence of the probe beams on the ATR prism 16. The incidence of the probe beams on the ATR prism 16 may be switched by controlling switching between on (emission) and off (non-emission) of a plurality of light sources. Alternatively, one light source for emitting light beams having a plurality of wavelengths may be used, and on and off of the light source may be switched on a wavelength-by-wavelength basis.

In the above-described embodiments, furthermore, as a non-limiting example, the first half mirror 131 and the second half mirror 132 are used as elements for transmitting some of the probe beams and reflecting the rest. Alternatively, a beam splitter, a polarization beam splitter, or the like may be used.

A high-refractive-index material that transmits probe beams, such as germanium, has a high surface reflectance in terms of material characteristics. For example, when light polarized in a direction perpendicular to the surface direction of the substrate (s-polarized light) is incident on the substrate at an incident angle of 45 degrees, the ratio of transmission to reflection is substantially 1:1. Using this, a germanium plate installed to provide an incident angle of 45 degrees may be used in place of a half mirror. Since the back surface also has a reflection component of 50%, a non-reflection preventing film is formed on the back surface.

### Modifications of Embodiments

Components in embodiments may be modified in various ways. The following describes various modifications.

Suppression of Influence of Linearity Error of Photodetector 17

The photodetector 17 in the blood glucose measurement apparatus 100 may include a linearity error, and the linearity error of the photodetector 17 causes a measurement error of the blood glucose level. The influence of the linearity error can be reduced by changing the light intensities of the probe beams in three or more stages determined in advance and comparing the light intensities of the probe beams with the detection values obtained by the photodetector 17.

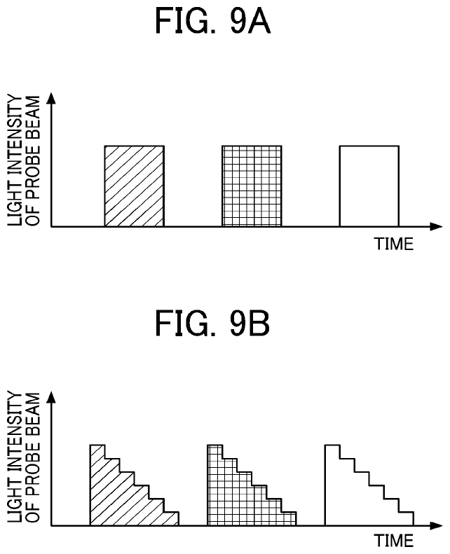

FIGS. 9A and 9B are diagrams for describing an example of the light intensities of the probe beams that are changed in three or more stages. FIG. 9A illustrates the light intensities of the probe beams according to a comparative example, and FIG. 9B illustrates the light intensities of the probe beams that are changed in three or more stages. In FIGS. 9A and 9B, a hatched portion represents the light intensity of the first probe beam, a cross-hatched portion represents the light intensity of the second probe beam, and a non-hatched portion represents the light intensity of the third probe beam.

In FIG. 9A, the light intensities of the respective probe beams are constant, whereas in FIG. 9B, the light intensities of the respective probe beams gradually decrease stepwise in three or more stages. Changing the drive voltage or drive current of a light source in three or more stages determined in advance (in FIG. 9B, six stages) can change the light intensity of the probe beam to be emitted from the light source in three or more stages. In this case, the light intensities of the probe beams change at intervals of a shorter cycle than the cycle in which the shutter control unit 214 controls the switching of the probe beams (for example, the cycle from steps S82 to S84 in FIG. 8).

When the photodetector 17 does not include the linearity error, detection values obtained by the photodetector 17 linearly change with a change in the light intensities of the probe beams. On the other hand, when the photodetector 17 includes the linearity error, detection values obtained by the photodetector 17 non-linearly change with a change in the light intensities of the probe beams.

Accordingly, the probe beams are emitted while the light intensities of the probe beams are changed in three or more stages, detection values obtained by the photodetector 17 are acquired in each stage, and the light intensities of the emitted probe beams and the detection values obtained by the photodetector 17 are compared to identify a light intensity range in which linearity is ensured. Then, the absorbance and the blood glucose level is measured using a portion in which linearity is ensured, out of the probe light intensities that change in three or more stages. This enables measurement of the absorbance and the blood glucose level while reducing the influence of the linearity error of the photodetector 17.

Identifying the light intensity range in which linearity is ensured may be performed prior to blood glucose measurement, or may be performed in real time during blood glucose measurement.

Since one photodetector 17 is used for a plurality of probe beams, the process of reducing the influence of the linearity error of the photodetector 17 using all of the plurality of probe beams may not be necessary, and is performed using at least one of the plurality of probe beams.

Detection of Probe Beam using Image Sensor

The photodetector 17 is not limited to one employing one pixel (light-receiving element). Alternatively, the photodetector 17 can be a line image sensor in which pixels are arranged in a line, or an area image sensor in which pixels are arranged two-dimensionally.

A detection signal of the photodetector 17 is an integrated value of the light intensity of a received probe beam. At the contact of the live subject S with the ATR prism 16, the optical path of incident light on the ATR prism 16 or the optical path of outgoing light from the ATR prism 16 may change. Therefore, a detection error may occur due to the integration of the light intensity of the probe beam before and after such a change. As a result, accurate absorbance data may not be obtained.

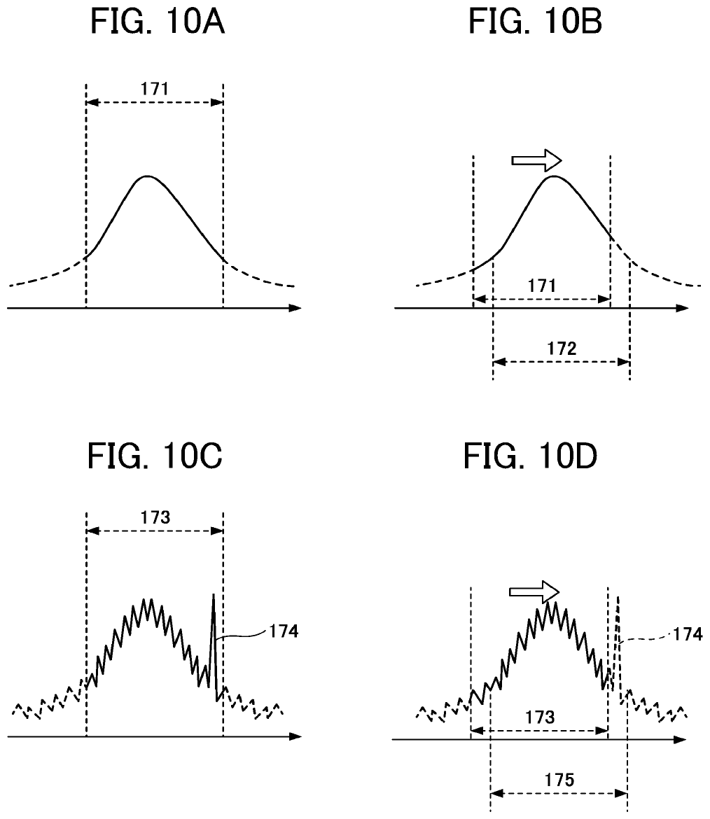

FIGS. 10A and 10B illustrate a displacement of a probe beam. FIG. 10A illustrates a range 171 that is a light-receiving range of the probe beam by the photodetector 17. When the probe beam displaces in a direction indicated by a hollow arrow in FIG. 10B, the light intensity distribution of the probe beam in the range 171 changes. As a result, the detection signal obtained by the photodetector 17 changes.

With the use of an image sensor as the photodetector 17, the amount of displacement of the probe beam is determined from a probe-beam image captured using the image sensor. Thus, the integrated value of the light intensity distribution of the probe beam after the displacement is used as the detection signal to correct the influence of the displacement of the probe beam. In FIG. 10B, a range 172 is a range where the integrated value of the light intensity distribution is acquired by using the probe beam after the displacement.

When coherent light such as laser light is used as the probe beam, a spot-like small light intensity distribution, called speckle, may be superimposed on the probe beam. FIG. 10C illustrates an example cross-sectional light intensity distribution of a probe beam including speckle. FIG. 10C illustrates a singular point 174 of the light intensity that may be included in a speckle image. The singular point 174 is included in a range 173.

FIG. 10D illustrates a displacement of the probe beam illustrated in FIG. 10C in a direction indicated by a hollow arrow. In this state, the singular point 174 is not included in the range 173, and the change in the detection signal before and after the displacement is significant. By contrast, when the integrated value of the light intensity distribution in a range 175 is used as the detection signal in accordance with the amount of displacement of the probe beam detected from the probe-beam image, the influence of the displacement of the probe beam can be more suitably corrected.

In addition, variations in measurement can be reduced as follows. Estimate a contact region between the live subject S and the ATR prism 16 based on the light intensity distribution of the probe beam on the image sensor, and correct the detection value based on the detection signal of the image sensor based on the sensitivity distribution in the surface of the ATR prism 16, which is acquired and stored in advance before the measurement is started.

Incidence Surface of Total Reflection Member

In the above-described embodiments, as a non-limiting example, the incidence surface 161 of the ATR prism 16 is a flat surface. The incidence surface 161 may be a surface having any shape, such as a diffusion surface or a surface having a curvature.

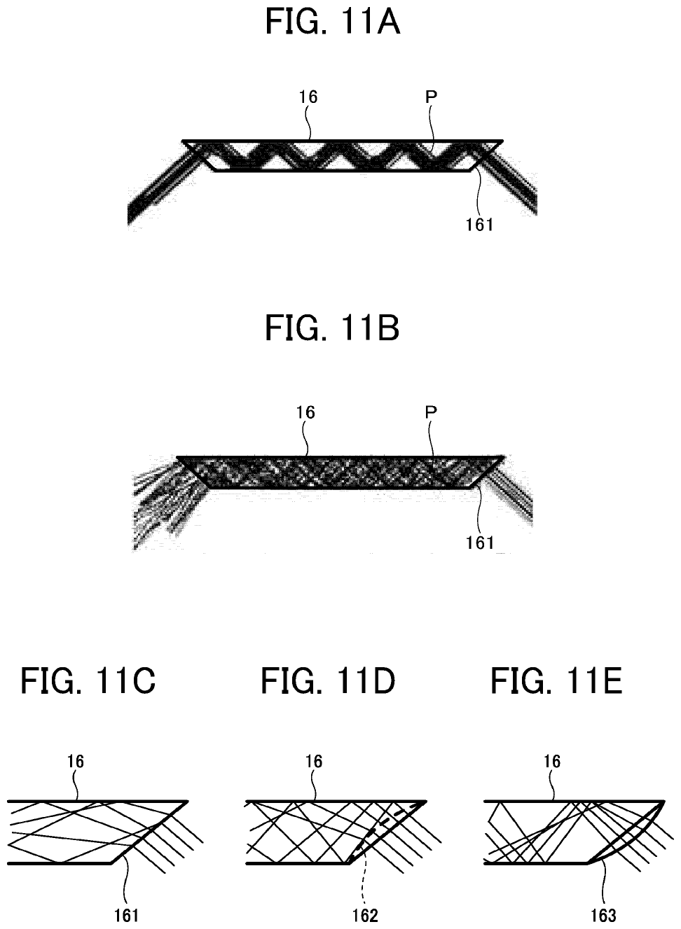

As illustrated in FIG. 11A, when the incidence surface 161 is a flat surface, the probe beams travel in a uniform direction in the ATR prism 16 in accordance with the incident angle on the incidence surface 161. Accordingly, in the total reflection surfaces of the ATR prism 16 with which the live subject S comes into contact, there may arise region dependence, that is, the measurement sensitivity differs for each region.

The detection signals of the photodetector 17 depend on the contact state such as the area of the contact of the live subject S with the ATR prism 16. In particular, when the live subject S, such as lips or a finger, is an object to be measured, the reproducibility of the contact state is likely to be low, and the measurement variation may increase due to the region dependence of the measurement sensitivity.

By contrast, when the incidence surface 161 is a diffusion surface, the traveling directions of the probe beams in the ATR prism 16 are randomly different. This structure can relax the region dependence of the measurement sensitivity and reduce the measurement variations as illustrated in FIG. 11B.

Examples of the incidence surface 161 include a diffusion surface illustrated in FIG. 11C, a concave surface illustrated in FIG. 11D, and a convex surface illustrated in FIG. 11E. The concave surface illustrated in FIG. 11D and the convex surface illustrated in FIG. 11E are examples of an incidence surface having a curvature. Like a diffusion surface, the concave surface and the convex surface can make the optical paths of the probe beams different, thereby relaxing the region dependence of the measurement sensitivity and reduce the measurement variation.

A configuration in which a diffusion plate, a lens, and the like are disposed along the optical path before the probe beams are incident on the ATR prism 16 also achieves a similar effect. In this case, however, the number of components of the apparatus increases, which may lead to an increase in cost or a difference in measurement value among apparatuses (differences among apparatuses) due to an assembly error. It is more preferable to form the incidence surface 161 of the ATR prism 16 as a diffusion surface or a curved surface since the differences among apparatuses or the increase in cost can be reduced.

Support for Light Guide and Total Reflection Member

When the live subject S comes into contact with the ATR prism 16, changes in relative position between the first hollow optical fiber 151 and the ATR prism 16 and changes in relative position between the second hollow optical fiber 152 and the ATR prism 16 may cause fluctuations in the incidence efficiency or emission efficiency of the probe beams with respect to the ATR prism 16, thereby increasing the measurement variation.

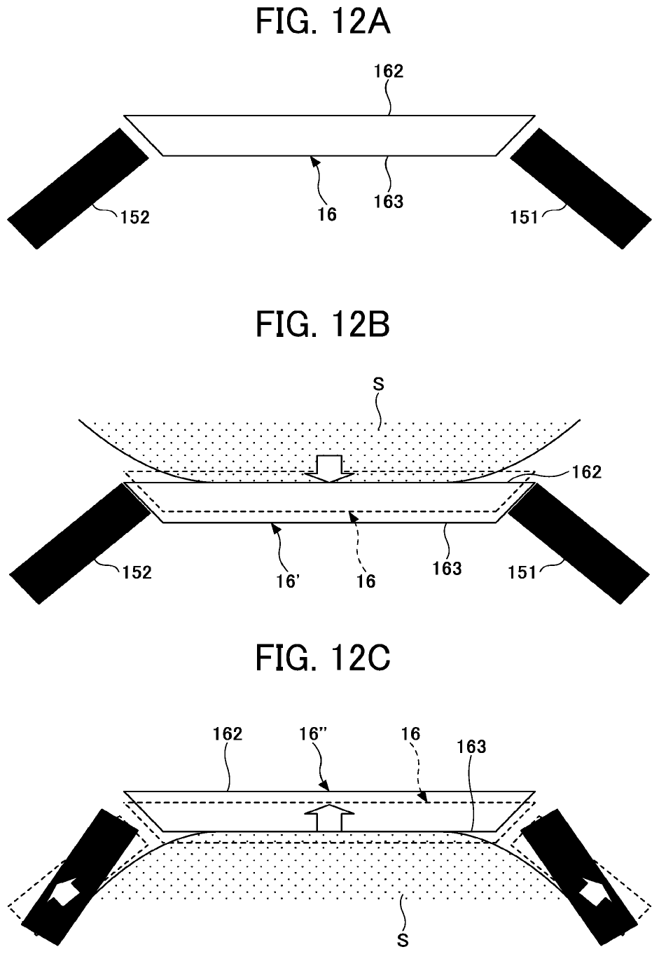

FIGS. 12A to 12C illustrate changes in relative position between the first hollow optical fiber 151 and the ATR prism 16 and changes in relative position between the second hollow optical fiber 152 and the ATR prism 16. FIG. 12A illustrates a case where the ATR prism 16 is not in contact with the live subject S, FIG. 12B illustrates a case where the live subject S is in contact with the first total reflection surface 162 of the ATR prism 16, and FIG. 12C illustrates a case where the live subject S is in contact with the second total reflection surface 163 of the ATR prism 16.

As illustrated in FIG. 12B, when the live subject S is in contact with the first total reflection surface 162 of the ATR prism 16, as indicated by a hollow arrow, a pressing force is applied downward, and the ATR prism 16 is displaced downward. As a result, the ATR prism 16 is at a displaced position 16′, and the relative position between the first hollow optical fiber 151 and the ATR prism 16 and the relative position between the second hollow optical fiber 152 and the ATR prism 16 are changed.

As illustrated in FIG. 12C, when the live subject S is in contact with the second total reflection surface 163 of the ATR prism 16, as indicated by a hollow arrow, a pressing force is applied upward, and the ATR prism 16 is displaced upward. As a result, an ATR prism 16″ at the displaced position is obtained, and the relative position between the first hollow optical fiber 151 and the ATR prism 16″ and the relative position between the second hollow optical fiber 152 and the ATR prism 16″ are changed.

Such changes in relative position cause fluctuations in the incidence efficiency or emission efficiency of the probe beams with respect to the ATR prism 16. In particular, when the object to be measured is a live subject, it is difficult to keep the contact pressure constant, and, in particular, the measurement variation due to the changes in relative position is likely to increase.

To suppress the changes in relative position, the first hollow optical fiber 151 and the ATR prism 16 are preferably supported by the same supporting member, and the second hollow optical fiber 152 and the ATR prism 16 are preferably supported by the same supporting member.

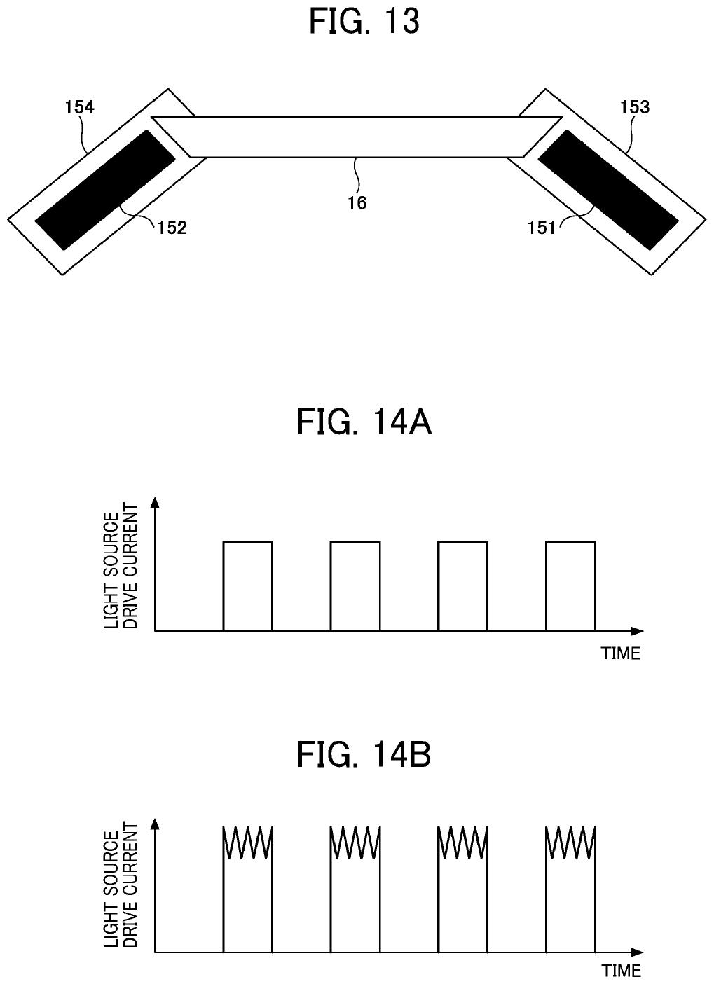

FIG. 13 illustrates an example configuration of members that support the first hollow optical fiber 151, the second hollow optical fiber 152, and the ATR prism 16. In FIG. 13, a light guide supporting member 153 is a member that integrally supports the first hollow optical fiber 151 and the ATR prism 16. A light emission supporting member 154 is a member that integrally supports the second hollow optical fiber 152 and the ATR prism 16.

The first hollow optical fiber 151 and the ATR prism 16, which are integrally supported, move together when the live subject S is brought into contact with the ATR prism 16. Thus, no relative position change occurs. The second hollow optical fiber 152 and the ATR prism 16, which are integrally supported, move together when the live subject S is brought into contact with the ATR prism 16. Thus, relative position does not change. As a result, the fluctuations in the incidence efficiency and emission efficiency of the probe beams, which are caused by a contact of the live subject S with the ATR prism 16, can be suppressed, and the measurement variation can be reduced.

In the example described above, the light guide supporting member 153 and the light emission supporting member 154 are separate members. Alternatively, the first hollow optical fiber 151, the second hollow optical fiber 152, and the ATR prism 16 may be supported by one supporting member.

Even in a case where the light guide is constructed of an optical element such as a mirror or a lens without using the first hollow optical fiber 151, an effect similar to that described above can be achieved by integrally supporting the optical element and the ATR prism 16.

In addition to the light guide, the first light source 111, the second light source 112, the third light source 113, and the photodetector 17 may also be integrally supported by the same supporting member. In this case, the effect of reducing the measurement variation can be achieved.

High-Frequency Modulation of Light Source Drive Current

When a probe beam includes speckle, the detection value obtained by the photodetector 17 may fluctuate in accordance with the pattern of the speckle, resulting in an increase in measurement variation. The speckle is generated by interference of scattered light or the like of the probe beam. Reducing the coherence of the probe beam can suppress the generation of the speckle. In the embodiments, the high-frequency modulation component is superimposed on the current for driving the light source, thereby reducing the coherence of the light source included in the blood glucose measurement apparatus and reduce the measurement variation of the absorbance caused by the speckle in the probe beam.

FIGS. 14A and 14B illustrate examples of the light source drive current. FIG. 14A illustrates a light source drive current according to a comparative example, and FIG. 14B illustrates a high-frequency modulated light source drive current.

The light source control unit 212 (see FIG. 6) periodically outputs a pulsed drive current illustrated in FIG. 14A to the first light source 111, the second light source 112, and the third light source 113 to cause the first light source 111, the second light source 112, and the third light source 113 to emit pulsed probe beams.

In embodiments, a high-frequency modulation component is superimposed on the pulsed drive current in FIG. 14A, and the resulting drive current is output to the first light source 111, the second light source 112, and the third light source 113. The high-frequency modulation component may have a sinusoidal or rectangular waveform. Any modulation frequency from 1 megahertz (MHz) to several gigahertz (GHz) is selectable.

The superimposition of the high-frequency modulation component allows each of the first light source 111, the second light source 112, and the third light source 113 to artificially emit a multimode laser beam as a probe beam, reducing the coherence of the probe beam.

The reduction in coherence reduces the speckle in the probe beam and reduces the measurement variation caused by the speckle.

### First Embodiment

Next, a blood glucose measurement apparatus according to a first embodiment will be described.

In this measurement apparatus, when an object to be measured such as the lips of the subject comes into contact with an optical device included in the apparatus, a change in the temperature of the object to be measured causes a change in absorption spectrum, which may reduce the reliability of measurement. In this embodiment, a contact region of a total reflection member configured to totally reflect an incoming probe beam in contact with an object to be measured, the contact region being in contact with the object to be measured, is maintained at a predetermined temperature using a temperature adjuster, thereby suppressing a temperature difference between the total reflection member and the object to be measured. Accordingly, when the object to be measured, such as the lips of the subject, comes into contact with the total reflection member, a change in absorption spectrum caused by a change in the temperature of the object to be measured is suppressed, and a reduction in measurement reliability is prevented.

Example Configuration of Blood Glucose measurement Apparatus

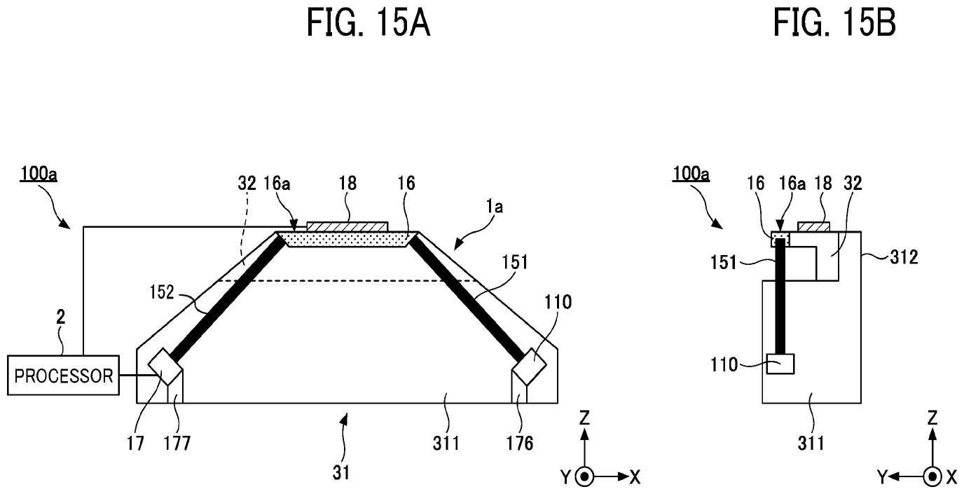

First, the configuration of a blood glucose measurement apparatus 100a according to this embodiment will be described. FIGS. 15A and 15B illustrate an example configuration of the blood glucose measurement apparatus 100a. FIG. 15A is a front view of the blood glucose measurement apparatus 100a, and FIG. 15B is a side view of the blood glucose measurement apparatus 100a.

As illustrated in FIGS. 15A and 15B, the blood glucose measurement apparatus 100a includes a measurement device 1a, and the measurement device 1a includes a first support 31, a second support 32, a quantum cascade laser (QCL) 110, and a planar heat generating element 18.

The first support 31 includes a hollow box-shaped member 311, and a back plate 312 disposed on a surface of the box-shaped member 311 on the positive Z direction side. The material of the box-shaped member 311 and the back plate 312 is not limited to any specific material.

In the box-shaped member 311, the QCL 110, the first hollow optical fiber 151, the second hollow optical fiber 152, and the photodetector 17 are supported. In FIGS. 15A and 15B, the inside of the box-shaped member 311 is illustrated in a see-through state.

The box-shaped member 311 has a bottom plate having a light source support 176 and a photodetector support 177 secured to a surface thereof on the positive Z direction side. The QCL 110 is secured to an inclined surface portion of the light source support 176, and the photodetector 17 is secured to an inclined surface portion of the photodetector support 177. The securing of the QCL 110 and the photodetector 17 may be performed by using an adhesive, screws, or the like. The same applies when the term “secured” is used in the following description.

The QCL 110 is a tunable quantum cascade laser and configured to emit a laser beam having a wave number of 1050 cm⁻¹ as a first probe beam, emit a laser beam having a wave number of 1070 cm⁻¹ as a second probe beam, and emit a laser beam having a wave number of 1100 cm⁻¹ as a third probe beam.

In other words, the QCL 110 has the functions of the first light source 111, the second light source 112, and the third light source 113 in FIG. 1. In this embodiment, the emission of the first to third probe beams from the QCL 110 can be switched by a control signal. Thus, members for switching wavelengths, such as the first shutter 121, the second shutter 122, the third shutter 123, the first half mirror 131, and the second half mirror 132 illustrated in FIG. 1, are omitted. In the following, the first to third probe beams are collectively referred to as probe beams P.

One end of the first hollow optical fiber 151 is secured to the QCL 110 in such a manner as to guide the probe beams P, and is supported by the QCL 110. A portion on the side of the first hollow optical fiber 151 connecting to the QCL 110 in the length direction is accommodated in the box-shaped member 311. The remaining portion of the first hollow optical fiber 151 protrudes from the box-shaped member 311 toward the ATR prism 16, and the other end of the first hollow optical fiber 151 corresponding to an end portion on the protruding side of the first hollow optical fiber 151 is abutted against the incidence surface 161 of the ATR prism 16. The other end of the first hollow optical fiber 151 is not secured to the ATR prism 16, and the ATR prism 16 can be separated from the first hollow optical fiber 151.

One end of the second hollow optical fiber 152 is secured to the photodetector 17 in such a manner as to guide the probe beams P, and is supported by the photodetector 17. A portion on the side of the second hollow optical fiber 152 connecting to the photodetector 17 in the length direction is accommodated in the box-shaped member 311, and the remaining portion of the second hollow optical fiber 152 protrudes from the box-shaped member 311 toward the ATR prism 16. The other end of the second hollow optical fiber 152 corresponding to an end portion on the protruding side of the second hollow optical fiber 152 is abutted against the emission surface 164 of the ATR prism 16. The other end of the second hollow optical fiber 152 is not secured to the ATR prism 16, and the ATR prism 16 can be separated from the other end of the second hollow optical fiber 152.

As illustrated in FIG. 15B, the second support 32 is a member having an L shape when viewed from the X direction side, and is formed of a metal material having high heat conductivity, such as aluminum. A distal end face of the L-shaped second support 32 on the negative Z direction side is abutted against a surface of the box-shaped member 311 on the positive Z direction side. A surface of the second support 32 on the negative Y direction side is abutted against a surface of the back plate 312 on the positive Y direction side. The second support 32 is secured to the first support 31 in the state described above. However, the second support 32 may be configured to be detachable from the first support 31.

A distal end face of the L-shaped second support 32 on the positive Y direction side is abutted against a surface of the ATR prism 16 on the negative Y direction side, and the ATR prism 16 is secured to the second support 32. The second support 32 supports the ATR prism 16 by securing a side surface of the ATR prism 16 in the manner described above. An upper surface 16a of the ATR prism 16 on the positive Z direction side is a portion with which the lips of a live subject serving as an object to be measured come into contact.

The planar heat generating element 18 is secured to a surface of the second support 32 on the positive Z direction side. The planar heat generating element 18 is an example of a temperature adjuster. An electrical current flows through a thin metal plate of the heat generating element 18, and the entire surface of the thin metal plate generates heat. The heat generated by the planar heat generating element 18 is transferred to the ATR prism 16 in contact with the second support 32 via the second support 32, and heats the ATR prism 16. The planar heat generating element 18, which generates heat, is configured such that the supply of power to the planar heat generating element 18 is interrupted when a predetermined upper-limit temperature is exceeded to ensure safety.

Operation and Effect of Planar Heat Generating Element

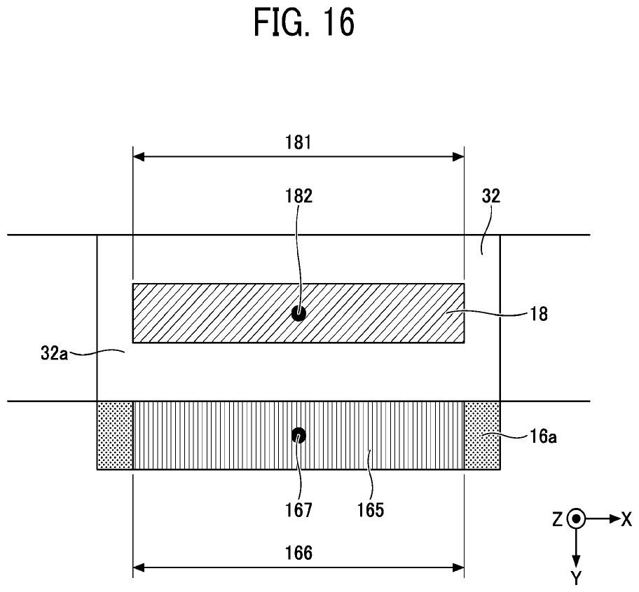

Next, referring to FIG. 16, the operation and effect of the planar heat generating element 18 will be described. FIG. 16 is a top view of the ATR prism 16 and peripheral portion thereof as viewed from the positive Z direction side, and illustrates a contact region between the ATR prism 16 and the lips of the subject.

In FIG. 16, the surface of the ATR prism 16 on the negative Y direction side is secured in contact with the distal end face of the L-shaped second support 32 on the positive Y direction side. Further, the planar heat generating element 18 is secured in contact with the surface of the second support 32 on the positive Z direction side.

The heat generated by the planar heat generating element 18 passes through a heat transfer portion 32a of the second support 32, which is disposed between the ATR prism 16 and the planar heat generating element 18, and heats the surface of the ATR prism 16 on the negative Y direction side. As a result, the temperature of the entire ATR prism 16 increases, and the temperature of the upper surface 16a of the ATR prism 16 can be increased accordingly.

The temperature of the ATR prism 16 is typically lower than the temperature (body temperature) of the live subject. For example, the temperature of the lips is about 33 degrees to 35 degrees, which is slightly lower than the body temperature, whereas the temperature of the ATR prism 16 is about 25 degrees, which corresponds to the outside air temperature. Accordingly, when the lips come into contact with the upper surface 16a of the ATR prism 16 for blood glucose measurement, the temperature of the lips on the contact portion may decrease due to heat exchange with the ATR prism 16, and the absorption spectrum may change. The change in absorption spectrum is not based on the blood glucose level, and becomes a measurement error for the blood glucose level acquired based on the absorption spectrum, resulting in a reduction in the reliability of measurement.

In this embodiment, in contrast, the heating by the planar heat generating element 18 increases the temperature of the contact region of the ATR prism 16 with the lips to equal to or higher than about 33 degrees and equal to or lower than about 35 degrees, which is substantially equal to the temperature of the lips. In addition, when the temperature of the ATR prism 16 changes due to a change in the outside air temperature or the like, the state of heating by the planar heat generating element 18 is maintained to maintain the temperature of the contact region of the ATR prism 16 with the lips at a predetermined temperature substantially equal to the temperature of the lips.

Accordingly, the temperature difference between the ATR prism 16 and the lips in the contact region can be suppressed, and the decrease in the temperature of the lips when the lips are in contact with the upper surface 16a of the ATR prism 16 can be suppressed. As a result, a measurement error caused by the decrease in the temperature of the lips can be prevented, and a reduction in the reliability of measurement can be prevented.

The predetermined temperature described above is preferably equal to or higher than 33 degrees and equal to or lower than 35 degrees, and in particular preferably 34 degrees. Note that values such as values equal to or higher than 33 degrees and equal to or lower than 35 degrees and 34 degrees do not require strict coincidence, and a difference which is typically recognized as an error is permissible.

In addition, heating the ATR prism 16 to a temperature higher than the outside air temperature can reduce the influence of dew condensation or the like caused by exhalation when the ATR prism 16 is held between the lips. This also can enhance the reliability of measurement.

In FIG. 16, a contact region 165 depicted as a vertically hatched portion indicates a contact region between the ATR prism 16 and the lips, and a contact length 166 indicates the length of the contact region 165 in the X direction. A heat generation length 181 indicates the length of the planar heat generating element 18 in the X direction.

It is preferable that the heat generation length 181 match the contact length 166. This can suppress the non-uniformity of heating on the ATR prism 16 in the X direction by the planar heat generating element 18. Accordingly, the temperature of the contact region 165 of the ATR prism 16 in the X direction can be uniformly maintained at a predetermined temperature, and the temperature difference between the upper surface 16a of the ATR prism 16 and the lips can be more accurately suppressed.

Note that the contact length 166 may vary due to a change in the manner in which the lips contact the ATR prism 16, or the contact length 166 may vary due to individual differences of live subjects. Accordingly, the heat generation length 181 and the contact length 166 do not necessarily exactly match, but are substantially the same.

The X direction is an example of a longitudinal direction. The contact length 166 is an example of the length of the contact region in the longitudinal direction, and the heat generation length 181 is an example of the length of the temperature adjuster in the longitudinal direction.

FIG. 16 illustrates a heat generation center 182 that is the center position of the planar heat generating element 18 in the X direction, and a contact center 167 that is the center position of the contact region 165 in the X direction. In the longitudinal direction of the heat generating element 18, the heat generation center 182 is preferably adjacent to the contact center 167, and more preferably, aligned with (matches) the contact center 167 in the X direction. With this configuration, as in the foregoing description, the temperature of the contact region 165 of the ATR prism 16 in the X direction can be uniformly maintained at a predetermined temperature, and the temperature difference between the upper surface 16a of the ATR prism 16 and the lips can be more accurately suppressed.

Note that, like the contact length 166 described above, the heat generation center 182 and the contact center 167 do not necessarily exactly match, but the heat generation center 182 and the contact center 167 have a predetermined positional relationship.

The heat generation center 182 is an example of an intermediate position of the temperature adjuster in the longitudinal direction, and the contact center 167 is an example of an intermediate position of the contact region in the longitudinal direction. The positional relationship between the heat generation center 182 and the contact center 167, which are aligned with each other in the X direction, is an example of a predetermined positional relationship.

In FIG. 16, by way of example, the contact region 165 is a portion of the upper surface 16a of the ATR prism 16 in the longitudinal direction. However, the entire upper surface 16a in the longitudinal direction may be set as the contact region 165, and the length of the planar heat generating element 18 in the longitudinal direction may be determined in accordance with the length of the contact region 165 in the X direction.

### Second Embodiment

Next, a blood glucose measurement apparatus 100b according to a second embodiment will be described.

In this embodiment, the planar heat generating element 18 is controlled based on the temperature detection value of the ATR prism 16 to more accurately maintain the contact region of the ATR prism 16 with the lips of the subject at a predetermined temperature.

Configuration of Blood Glucose Measurement Apparatus

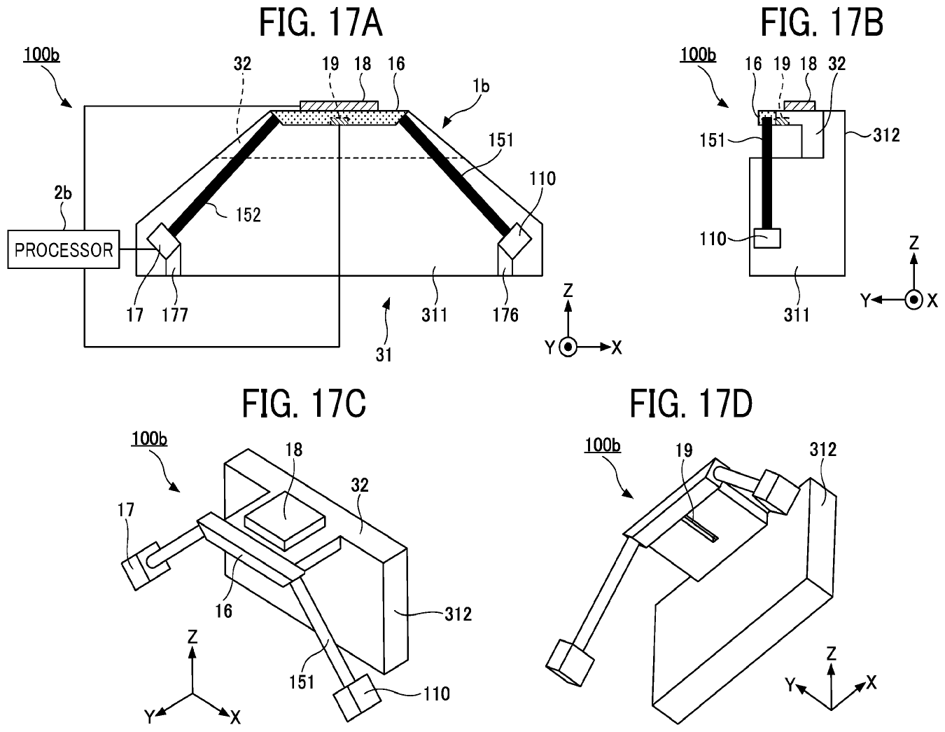

FIGS. 17A to 17D illustrate an example configuration of the blood glucose measurement apparatus 100b. FIG. 17A is a front view of the blood glucose measurement apparatus 100b, and FIG. 17B is a side view of the blood glucose measurement apparatus 100b, FIG. 17C is a perspective view of the ATR prism 16 and peripheral portion thereof as viewed from the positive X and positive Z directions, and FIG. 17D is a perspective view of the ATR prism 16 and peripheral portion thereof as viewed from the positive X and negative Z directions.

As illustrated in FIGS. 17A to 17D, the blood glucose measurement apparatus 100b includes a measurement device 1b and a processor 2b. The measurement device 1b includes a temperature sensor 19.

The temperature sensor 19 is a small sensor formed of a thermocouple, a thermistor, a resistance thermometer, or the like, and is disposed so as to be fitted into a recess in the face of the second support 32 that comes into contact with the ATR prism 16. The temperature sensor 19 is capable of detecting the temperature of the ATR prism 16 in contact with a portion of the surface of the ATR prism 16 on the negative Y direction side, and outputting a temperature detection value to the processor 2b, which is electrically connected to the temperature sensor 19. The temperature sensor 19 is an example of a “temperature detector”.

As described above, the planar heat generating element 18 is disposed on the surface of the second support 32 on the positive Z direction side. In other words, the temperature sensor 19 and the planar heat generating element 18 are held by the same holder. This can simplify the apparatus configuration. In addition, when the temperature of the ATR prism 16 is controlled via the holder of the planar heat generating element 18, the temperature sensor 19 can accurately detect the temperature of the holder. The second support 32 is an example of the holder.

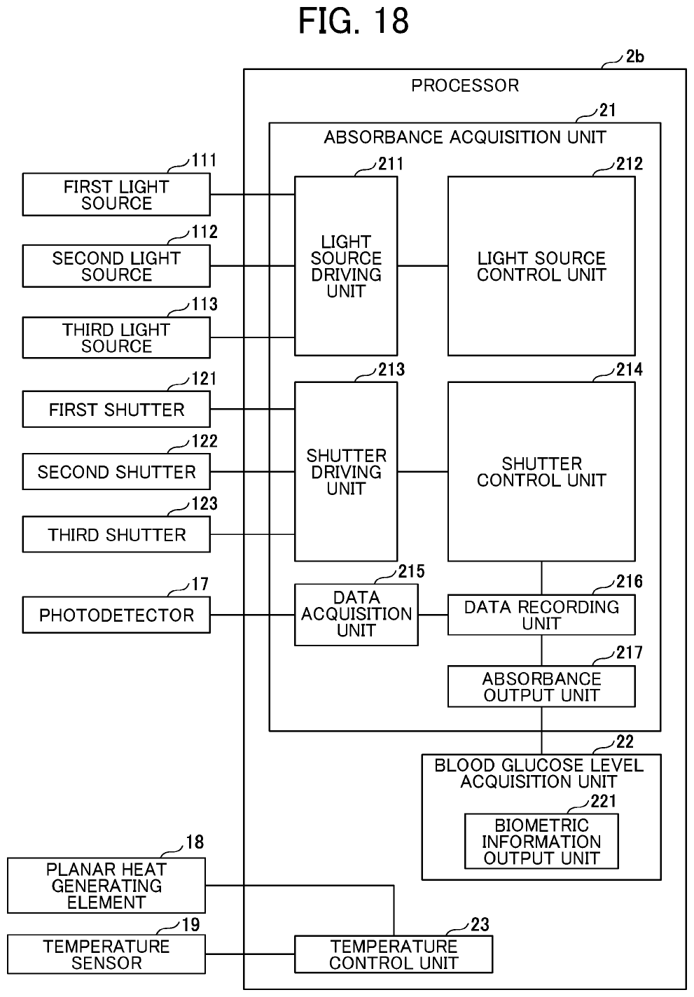

FIG. 18 is a block diagram illustrating an example functional configuration of the processor 2b. As illustrated in FIG. 18, the processor 2b includes a temperature control unit 23. The function of the temperature control unit 23 can be implemented by, for example, the CPU 501 illustrated in FIG. 5 executing a predetermined program.

The temperature control unit 23 has a function of controlling the planar heat generating element 18 based on a temperature detection value input from the temperature sensor 19. More specifically, the temperature control unit 23 outputs a control signal to the planar heat generating element 18 based on the temperature detection value input from the temperature sensor 19 to control heat generation of the planar heat generating element 18. Accordingly, the temperature of the contact region 165 of the ATR prism 16 (see FIG. 16) is controlled so as to suppress the temperature difference between the ATR prism 16 and the lips in the contact region 165. The control using the temperature control unit 23 may be implemented by a proportional integral differential (PID) control method or the like.

Example Temperature Control

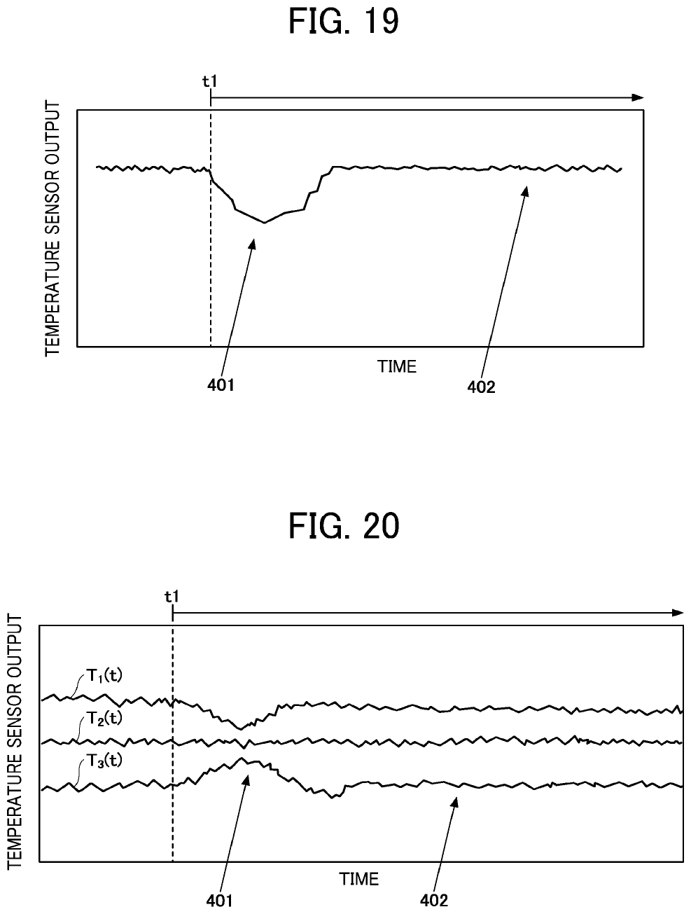

Next, referring to FIGS. 19 and 20, an example of temperature control results obtained by the processor 2b will be described. FIGS. 19 and 20 illustrate changes in the output of the temperature sensor 19 over time. FIG. 19 illustrates a case where this embodiment is not applied, and FIG. 20 illustrates a case where this embodiment is applied. In FIGS. 19 and 20, time t1 indicates a timing at which the lips start to come into contact with the upper surface 16a of the ATR prism 16. After the time t1, the lips are kept in contact with the upper surface 16a of the ATR prism 16.

As illustrated in FIG. 19, the output of the temperature sensor 19 decreases in a time region 401 immediately after the time t1, and is then stable in a time region 402.

Immediately after the time t1, due to a temperature difference between the ATR prism 16 and the lips in the contact region 165, heat transfers between the ATR prism 16 and the lips. Accordingly, the output of the temperature sensor 19 largely changes. After that, the heat transfer between the ATR prism 16 and the lips stops, and the output becomes stable.

In the time region 401, the temperature of contact regions of the lips with the ATR prism 16 also changes, and the absorption spectrum changes accordingly, resulting in an increase in the measurement error of the blood glucose level.

In FIG. 20, a line T₁(t) indicates changes in the output of the temperature sensor 19 over time when the temperature of the contact region 165 of the ATR prism 16 is controlled to 35 degrees. Likewise, a line T₂(t) indicates changes in the output of the temperature sensor 19 over time when the temperature of the contact region 165 is controlled to 34 degrees, and a line T₃(t) indicates changes in the output of the temperature sensor 19 over time when the temperature of the contact region 165 is controlled to 33 degrees.

As indicated by all of the lines T₁(t) to T₃(t), the changes in the output of the temperature sensor 19 over time immediately after the time t1 are suppressed compared with the case where this embodiment is not applied. This indicates that blood glucose measurement is performed, with the measurement error suppressed immediately after the time t1. In particular, as indicated by the line T₂(t) depicting the case where the temperature of the contact region 165 is controlled to 34 degrees, the output of the temperature sensor 19 does not substantially change. This indicates that it is preferable, in particular, to control the temperature of the contact region 165 to 34 degrees.

Operation and Effect of Processor

As described above, in this embodiment, the temperature control unit 23 controls the planar heat generating element 18 based on the temperature detection value of the ATR prism 16 obtained by the temperature sensor 19, thereby more accurately maintaining a contact region of the ATR prism 16 with the lips of the subject at a predetermined temperature. Accordingly, the temperature difference between the ATR prism 16 and the lips in the contact region can be suppressed, and the decrease in the temperature of the lips when the lips are in contact with the upper surface 16a of the ATR prism 16 can be suppressed. As a result, a measurement error caused by the decrease in the temperature of the lips can be prevented, and a reduction in the reliability of measurement can be prevented.

In this embodiment, furthermore, the temperature sensor 19 and the planar heat generating element 18 are held by the same holder. This can simplify the apparatus configuration of the blood glucose measurement apparatus 100b. In addition, when the temperature of the ATR prism 16 is controlled via the holder of the planar heat generating element 18, the temperature sensor 19 can accurately detect the temperature of the holder.

In the example described above, as a non-limiting example, the temperature sensor 19 detects the temperature of the ATR prism 16 in contact with the ATR prism 16. The temperature sensor 19 may not necessarily be in contact with the ATR prism 16 so long as the temperature of the contact region 165 of the ATR prism 16 can be detected. The temperature sensor 19 may be arranged at any position.

### Third Embodiment

Next, a blood glucose measurement apparatus 100c according to a third embodiment will be described.

In this embodiment, when the lips of the subject are brought into contact with the ATR prism 16 for blood glucose measurement, the lips are prevented from coming into contact with the planar heat generating element 18 that is generating heat to provide safety blood glucose measurement.

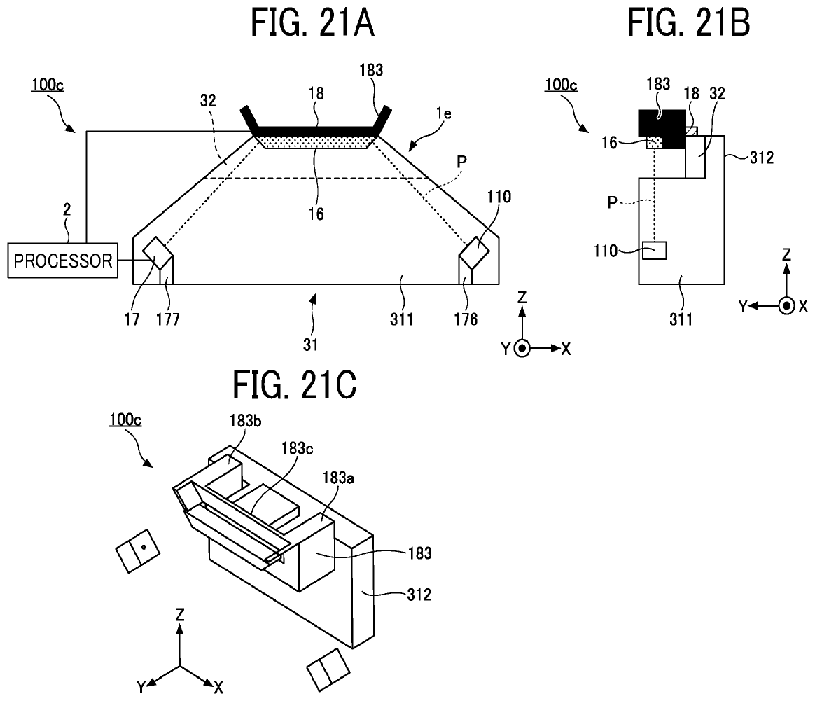

FIGS. 21A to 21C illustrate an example configuration of the blood glucose measurement apparatus 100c. FIG. 21A is a front view of the blood glucose measurement apparatus 100c, FIG. 21B is a side view of the blood glucose measurement apparatus 100c, and FIG. 21C is a perspective view of the ATR prism 16 and peripheral portion thereof as viewed from the positive X and positive Z directions. As illustrated in FIGS. 21A to 21C, the blood glucose measurement apparatus 100c includes a contact prevention member 183.

In the contact prevention member 183, walls 183a, 183b, and 183c are integral such that the wall 183a is on the positive X direction side of the planar heat generating element 18, the wall 183b is on the negative X direction side of the planar heat generating element 18, and the wall 183c is on the positive Y direction side of the planar heat generating element 18. The height of each of the walls 183a, 183b, and 183c (the length in the Z direction) is greater than the height of the planar heat generating element 18. This configuration prevents the lips from coming into contact with the planar heat generating element 18 even when the lips are brought close to the upper surface 16a of the ATR prism 16 from the positive Z direction side.

As described above, the walls 183a, 183b, and 183c of the contact prevention member 183 function as a spacer for preventing an object from entering the space around the planar heat generating element 18. Accordingly, when the lips are brought into contact with the ATR prism 16, the lips can be prevented from coming into contact with the planar heat generating element 18 that is generating heat. Accordingly, blood glucose measurement can be safely performed.

Modifications

The following describes various modifications of the embodiments.

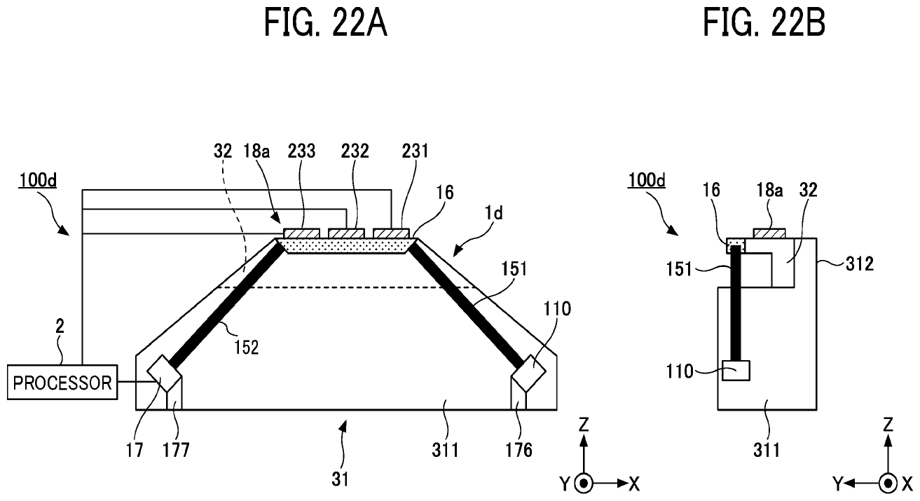

FIGS. 22A and 22B illustrate an example configuration of a blood glucose measurement apparatus 100d according to a first modification. FIG. 22A is a front view of the blood glucose measurement apparatus 100d, and FIG. 22B is a side view of the blood glucose measurement apparatus 100d. As illustrated in FIGS. 22A and 22B, the blood glucose measurement apparatus 100d includes a measurement device 1d, and the measurement device 1d includes a planar heat generating element 18a. The planar heat generating element 18a is constructed of three heat generators 231, 232, and 233. The heat generators 231, 232, and 233 are arranged at different positions in the X direction and are secured to the surface of the second support 32 on the positive Z direction side.

As described above, the configuration of the planar heat generating element 18a in which the plurality of small heat generators 231, 232, and 233 are arranged at different positions in the X direction can reduce the temperature difference of the ATR prism 16 in the X direction.

The reduction in the size of the heat generators is advantageous in returning the temperature of the ATR prism 16 to a target control value in a shorter time when the temperature of the ATR prism 16 changes immediately after the lips of the subject come into contact with the ATR prism 16.

When the blood glucose measurement apparatus 100d has a sleep mode as an operation mode, the blood glucose measurement apparatus 100d can recover from the sleep mode in a short time with a small overshoot to follow the target control value.

The sleep mode is a low-power-consumption operation mode in which the power consumption of the blood glucose measurement apparatus 100d is reduced. For example, if no data or signal is input within a predetermined time, the supply of power to the blood glucose measurement apparatus 100d is stopped to make a transition of the operation mode of the blood glucose measurement apparatus 100b to the sleep mode.

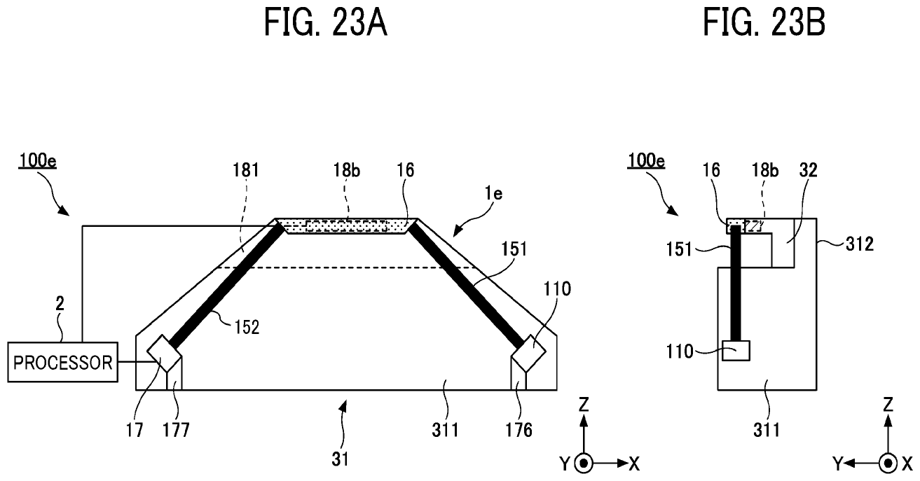

FIGS. 23A and 23B illustrate an example configuration of a blood glucose measurement apparatus 100e according to a second modification. FIG. 23A is a front view of the blood glucose measurement apparatus 100e, and FIG. 23B is a side view of the blood glucose measurement apparatus 100e. As illustrated in FIGS. 23A and 23B, the blood glucose measurement apparatus 100e includes a measurement device 1e, and the measurement device 1e includes a planar heat generating element 18b. The planar heat generating element 18b is secured so as to be fitted in a recess in a face of the L-shaped second support 32 on the positive Y direction side in contact with the surface of the ATR prism 16 on the negative Y direction side.

As described above, a planar heat generating element may be secured at any position so long as the planar heat generating element is capable of heating the ATR prism 16 and maintaining the temperature of the contact region 165 at a predetermined temperature. The planar heat generating element may be configured to come into contact with the ATR prism 16.

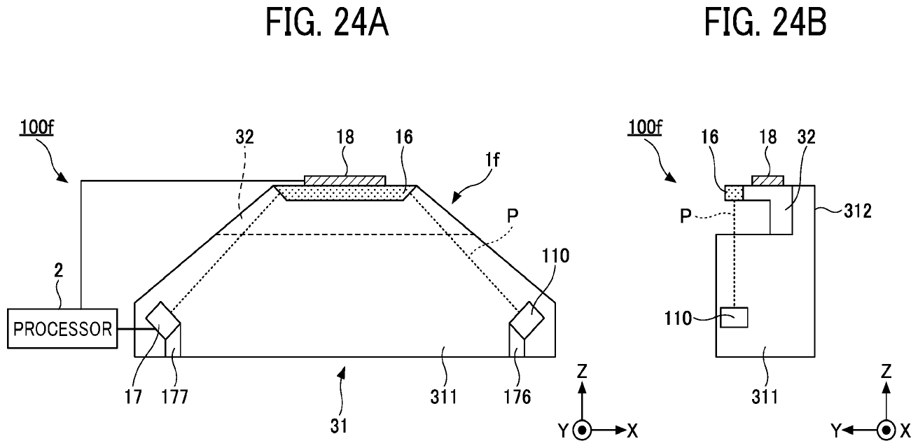

FIGS. 24A and 24B illustrate an example configuration of a blood glucose measurement apparatus 100f according to a third modification. FIG. 24A is a front view of the blood glucose measurement apparatus 100f, and FIG. 24B is a side view of the blood glucose measurement apparatus 100f. As illustrated in FIGS. 24A and 24B, the blood glucose measurement apparatus 100f does not include the first hollow optical fiber 151 or the second hollow optical fiber 152. The probe beams emitted from the QCL 110 are incident on the ATR prism 16 without the intervention of a light guide member such as the first hollow optical fiber 151. The probe beams emerging from the ATR prism 16 are incident on the photodetector 17 without the intervention of a light guide member such as the second hollow optical fiber 152. The blood glucose measurement apparatus 100f may have the configuration described above.

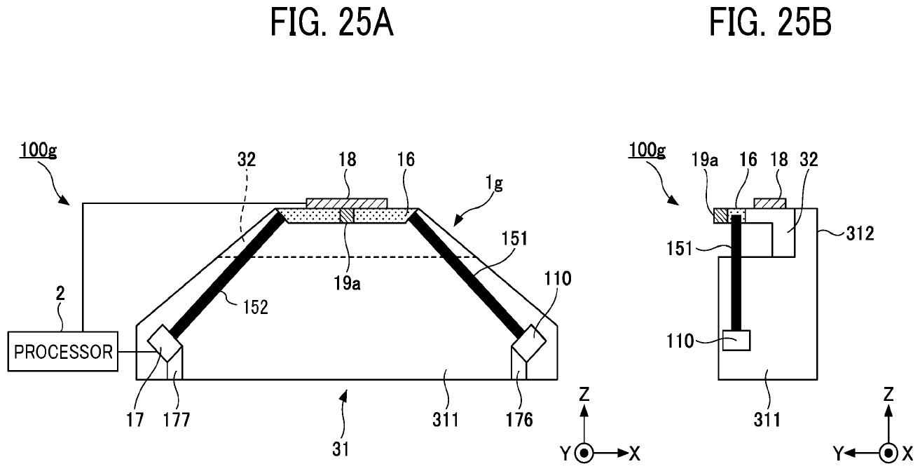

Next, referring to FIGS. 25 to 27, modifications of the temperature sensor 19 will be described.

FIGS. 25A and 25B illustrate an example configuration of a blood glucose measurement apparatus 100g according to a fourth modification. FIG. 25A is a front view of the blood glucose measurement apparatus 100g, and FIG. 25B is a side view of the blood glucose measurement apparatus 100g. As illustrated in FIGS. 25A and 25B, the blood glucose measurement apparatus 100g includes a measurement device 1g, and the measurement device 1g includes a temperature sensor 19a. The temperature sensor 19a is secured to a surface of the ATR prism 16 on the positive Y direction side.

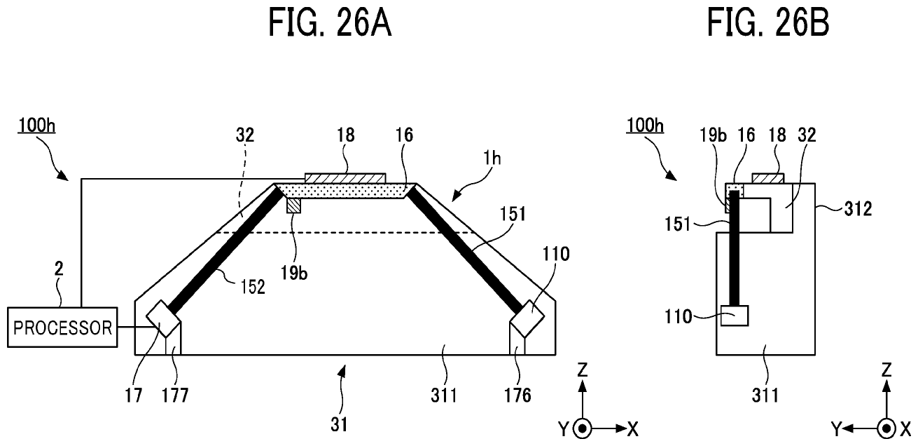

FIGS. 26A and 26B illustrate an example configuration of a blood glucose measurement apparatus 100h according to a fifth modification. FIG. 26A is a front view of the blood glucose measurement apparatus 100h, and FIG. 26B is a side view of the blood glucose measurement apparatus 100h. As illustrated in FIGS. 26A and 26B, the blood glucose measurement apparatus 100h includes a measurement device 1h, and the measurement device 1h includes a temperature sensor 19b. The temperature sensor 19b is secured to a surface of the ATR prism 16 on the negative Z direction side in the vicinity of an end portion of the ATR prism 16 on the negative X direction side.

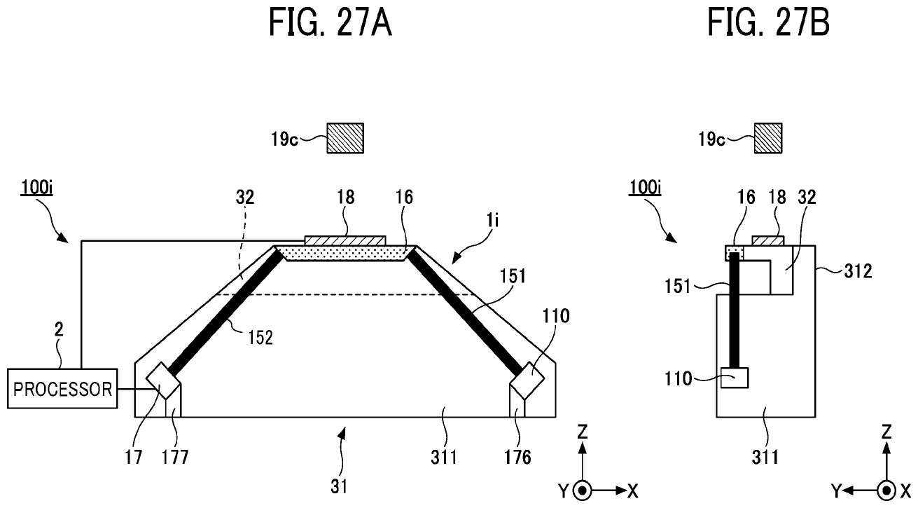

FIGS. 27A and 27B illustrate an example configuration of a blood glucose measurement apparatus 100i according to a sixth modification. FIG. 27A is a front view of the blood glucose measurement apparatus 100i, and FIG. 27B is a side view of the blood glucose measurement apparatus 100i. As illustrated in FIGS. 27A and 27B, the blood glucose measurement apparatus 100i includes a measurement device 1i, and the measurement device Ii includes a temperature sensor 19c. The temperature sensor 19c is a temperature sensor such as a radiation thermometer capable of detecting the temperature of an object to be examined in a contactless manner.

As described above, the arrangement of the temperature sensor 19 may be modified variously, and the temperature sensor 19 may be a contactless temperature sensor.

The above-described embodiments are illustrative and do not limit the present disclosure. Thus, numerous additional modifications and variations are possible in light of the above teachings. For example, elements and/or features of different illustrative embodiments may be combined with each other and/or substituted for each other within the scope of the present disclosure.

In the above-described embodiments, the planar heat generating element 18 is an example of a temperature adjuster. However, the temperature adjuster is not limited to the planar heat generating element 18 if the temperature adjuster is capable of maintaining the temperature of the upper surface 16a of the ATR prism 16 at a predetermined temperature. The temperature adjuster may be any other heat generating element such as a ceramic heater or a halogen heater. The temperature adjuster is preferably a small heat generating element that can be disposed adjacent to the ATR prism 16.

When the outside air temperature is high and the temperature of the ATR prism 16 is higher than the temperature of the lips of the subject, a cooling element may be disposed instead of a heat generating element as the temperature adjuster. Examples of the cooling element include a Peltier element. A heat generating element and a cooling element may be used in combination to perform temperature adjustment, or both the heating function and cooling function of a Peltier element may be utilized to perform temperature adjustment.

In the above-described embodiments, as a non-limiting example, the blood glucose level is measured. One or more embodiments are applicable to measurement of any other biometric information or non-biometric information if the measurement is based on the ATR method. In the example described above, the lips are brought into contact with the ATR prism 16. Alternatively, a portion other than the lips may be brought into contact with the ATR prism 16 for measurement.

In the case of a live subject, the predetermined temperature of a contact region maintained by the temperature adjuster such as the planar heat generating element 18 is set to the temperature of the live subject or a temperature of 33 to 35 degrees. In the case of an object to be measured other than a live subject, the predetermined temperature may be set to the temperature of a position at or adjacent to the surface of the object to be measured.

In the above-described embodiments, as a non-limiting example, the functions of the absorbance acquisition unit 21, the blood glucose level acquisition unit 22, the temperature control unit 23, and the like are implemented by the processor 2. These functions may be implemented by separate processors, or the functions of the absorbance acquisition unit 21 and the blood glucose level acquisition unit 22 may be implemented by a plurality of processors in a distributed manner. In addition, the functions of processors and the function of a storage device such as the data recording unit 216 may be implemented by an external device such as a cloud server.

Alternatively, an optical element such as a beam splitter that splits some of the probe beams emitted from a light source or emerging from a hollow optical fiber, and a detection element that detects the light intensities of the split probe beams may be disposed, and feedback control of the drive voltage or drive current of the light source may be performed to suppress the fluctuations in the light intensities of the probe beams. This configuration can suppress the fluctuations in the output of the light source and enables more accurate measurement of biometric information.

In addition, one or more embodiments are also applicable to a blood glucose measurement apparatus including one light source and configured to perform measurement by causing the light source to emit a probe beam having one wavelength.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), a digital signal processor (DSP), a field programmable gate array (FPGA), and conventional circuit components arranged to perform the recited functions.

1. A measurement apparatus comprising:
a total reflection member configured to totally reflect an incoming probe beam in a state being in contact with a measured object; and
a temperature adjuster configured to maintain, to a predetermined temperature, a temperature of a contact region of the total reflection member with the measured object.

2. The measurement apparatus according to claim 1, further comprising:
a light source configured to emit the probe beam;
a light intensity detector configured to detect a light intensity of the probe beam emerging from the total reflection member; and
circuitry configured to output a measurement value acquired based on the light intensity.

3. The measurement apparatus according to claim 1,
wherein the temperature adjuster is configured to adjust a temperature of the total reflection member to adjust the temperature of the contact region.

4. The measurement apparatus according to claim 1,
wherein a length of the temperature adjuster in a longitudinal direction of the temperature adjuster matches a length of the contact region in the longitudinal direction.

5. The measurement apparatus according to claim 1,
wherein, in a longitudinal direction of the temperature adjuster, an intermediate position of the temperature adjuster is adjacent to an intermediate position of the contact region.

6. The measurement apparatus according to claim 1,
wherein the predetermined temperature is a temperature of the measured object adjacent to a surface of the measured object.

7. The measurement apparatus according to claim 1,
wherein the predetermined temperature is a temperature of a live subject being the measured object.

8. The measurement apparatus according to claim 1,
wherein the predetermined temperature is equal to or higher than 33 degrees and equal to or lower than 35 degrees.

9. The measurement apparatus according to claim 1, further comprising:
a temperature sensor configured to detect a temperature of the total reflection member; and
circuitry configured to control the temperature adjuster based on a detection value obtained by the temperature sensor.

10. The measurement apparatus according to claim 9, further comprising a holder configured to hold the temperature sensor and the total reflection member.

11. The measurement apparatus according to claim 9, further comprising a contact prevention member configured to prevent contact between the temperature adjuster and the measured object.

12. A biometric information measurement apparatus comprising:
a total reflection member configured to totally reflect an incoming probe beam in a state being in contact with a live subject to be measured; and
a temperature adjuster configured to maintain, to a predetermined temperature, a temperature of a contact region of the total reflection member with the live subject.
